(19) United States
(12) Patent Application Publication
(10) Pub. No.: US 2007/0212301 A1
(43) Pub. Date: Sep. 13, 2007

(54) Monkey Immunoglobulin Sequences

(75) Inventors: Teri Aldrich, Seattle, WA (US); Wenyan Shen, Palo Alto, CA (US); Frederick W. Jacobsen, Newbury Park, CA (US); Arvia E. Morris, Seattle, WA (US); Martin J. Allen, Seattle, WA (US)

Correspondence Address: AMGEN INC.;LAW DEPARTMENT, 1201 AMGEN COURT WEST, SEATTLE, WA, 98119, US

(73) Assignee: AMGEN, INC., Thousand Oaks, CA (US)

(21) Appl. No.: 10/578,580

(22) PCT Filed: Nov. 4, 2004
(86) PCT No.: PCT/US04/37241

§ 371 Date: Mar. 21, 2007

Related U.S. Application Data
(60) Provisional application No. 60/517,970, filed on Nov. 7, 2003.

(51) Int. Cl. A61K 49/00; C07H 21/04; C07K 16/00; C12N 15/00; C12N 5/00; C12P 21/00

(52) U.S. Cl. 424/9.2; 435/320.1; 435/325; 435/69.1; 435/69.6; 530/300; 530/387.1; 536/23.53

(57) ABSTRACT

Nucleotide sequences encoding, and amino acid sequences comprising, heavy and light chain constant regions derived from cynomolgus monkeys. Chimeric antibodies with cynomolgus monkey constant regions are described.

### CROSS-REFERENCE TO RELATED APPLICATION

This application claims the benefit of U.S. provisional application No. 60/517,970, filed Nov. 7, 2003.

### FIELD

The present invention relates to monkey immunoglobulin sequences.

### BACKGROUND

Monkeys are used in evaluating antibodies. For example, lower primates such as monkeys often provide an animal model for studying diseases. In those instances where a monkey is used to study disease, antibodies may be introduced to determine their efficacy in treating or curing the disease. In certain instances, the antibodies being tested are from another species.

Like any other foreign antigen, an introduced, foreign antibody can trigger a monkey's immune system to mount a response against the antibody. For example, humans who receive antibodies generated in mice may develop an immune response to the mouse antibodies (Exley A. R. et al., Lancet 335:1275-77 (1990)). Likewise, in certain instances, a monkey can develop antibodies to the antibody from another species that is being tested. The monkey's immune response to foreign antibodies may inhibit their function, thus impeding the evaluation of the foreign antibodies.

Chimeric antibodies, containing amino acid sequences from more than one species, may in certain instances reduce the immune response a host would mount against the chimeric antibody, as compared to the host's immune response to an antibody that contains amino acid sequences only from a species different from the hosts species. For example, as discussed above, humans may mount an immune response to mouse antibodies. When part of the mouse antibody amino acid sequence is replaced with human antibody sequence, the human's immune response to the resulting chimeric antibody may be reduced (LoBuglio A. F. et al., PNAS-USA 86:4220-24 (1989)).

### SUMMARY OF THE INVENTION

In certain embodiments, an isolated polypeptide is provided comprising an amino acid sequence as set forth in SEQ ID NO:6; SEQ ID NO:8; SEQ ID NO:10; SEQ ID NO:12; SEQ ID NO:14; or SEQ ID NO:20 and further comprising an antibody heavy chain variable region.

In certain embodiments, an isolated polypeptide is provided comprising an amino acid sequence as set forth in SEQ ID NO:30.

In certain embodiments, an isolated polynucleotide is provided comprising a sequence encoding a polypeptide comprising an amino acid sequence as set forth in SEQ ID NO:6; SEQ ID NO:8; SEQ ID NO:10; SEQ ID NO:12; SEQ ID NO:14; or SEQ ID NO:20 and further comprising a sequence encoding a polypeptide comprising an antibody heavy chain variable region.

In certain embodiments, an isolated polynucleotide is provided comprising a sequence encoding a polypeptide comprising an amino acid sequence as set forth in SEQ ID NO:30 and further comprising a sequence encoding a polypeptide comprising an antibody light chain variable region.

In certain embodiments, an isolated antibody is provided comprising an amino acid sequence as set forth in SEQ ID NO:6; SEQ ID NO:8; SEQ ID NO:10; SEQ ID NO:12; SEQ ID NO:14; or SEQ ID NO:20 and a polypeptide comprising an amino acid sequence as set forth in SEQ ID NO:30.

In certain embodiments, a method for making a polypeptide is provided.

In certain embodiments, a method for making a chimeric antibody is provided.

In certain embodiments, a method for evaluating the effects of an antibody is provided comprising:
a) introducing into a cynomolgus monkey a chimeric antibody comprising light chain and heavy chain variable regions from an antibody and light chain and heavy chain constant regions from a cynomolgus monkey; and
b) evaluating the effects of the chimeric antibody in the cynomolgus monkey.

### BRIEF DESCRIPTION OF THE FIGURES

FIG. 1 shows the cDNA nucleotide sequence encoding the cyno3-16 cynomolgus monkey heavy chain constant region (SEQ ID NO. 1) and the amino acid sequence of the cyno3-16 cynomolgus monkey heavy chain constant region (SEQ ID NO. 2).

FIG. 2 shows the genomic DNA nucleotide sequence encoding the cyno33 cynomolgus monkey heavy chain constant region (SEQ ID NO. 3) and the amino acid sequence of the cyno33 cynomolgus monkey heavy chain constant region (SEQ ID NO. 4).

FIG. 3 shows the genomic nucleotide sequence encoding the cyno2-4 cynomolgus monkey heavy chain constant region (SEQ ID NO. 5) and the amino acid sequence of the cyno2-4 cynomolgus monkey heavy chain constant region (SEQ ID NO. 6).

FIG. 4 shows the genomic nucleotide sequence encoding the cyno2-4cys cynomolgus monkey heavy chain constant region (SEQ ID NO. 7) and the amino acid sequence of the cyno2-4cys cynomolgus monkey heavy chain constant region (SEQ ID NO. 8).

FIG. 5 shows the genomic nucleotide sequence encoding the cynods1 cynomolgus monkey heavy chain constant region (SEQ ID NO. 9) and the amino acid sequence of the cynods1 cynomolgus monkey heavy chain constant region (SEQ ID NO. 10).

FIG. 6 shows the cDNA nucleotide sequence encoding the cyno439 cynomolgus monkey heavy chain constant region (SEQ ID NO. 11) and the amino acid sequence of the cyno439 cynomolgus monkey heavy chain constant region (SEQ ID NO. 12).

FIG. 7 shows the cDNA nucleotide sequence encoding the cyno686 cynomolgus monkey heavy chain constant region (SEQ ID NO. 13) and the amino acid sequence of the cyno686 cynomolgus monkey heavy chain constant region (SEQ ID NO. 14).

FIG. 8 shows the genomic nucleotide sequence encoding the cyno35 cynomolgus monkey heavy chain constant region (SEQ ID NO.15) and the amino acid sequence of the cyno35 cynomolgus monkey heavy chain constant region (SEQ ID NO. 16 ).

FIG. 9 shows the genomic nucleotide sequence encoding the cyno36 cynomolgus monkey heavy chain constant region (SEQ ID NO.17) and the amino acid sequence of the cyno36 cynomolgus monkey heavy chain constant region (SEQ ID NO.18).

FIG. 10 shows the cDNA nucleotide sequence encoding the cyno477 cynomolgus monkey heavy chain constant region (SEQ ID NO. 19) and the amino acid sequence of the cyno477 cynomolgus monkey heavy chain constant region (SEQ ID NO. 20).

FIG. 11 shows the genomic nucleotide sequence encoding the cyno32 cynomolgus monkey heavy chain constant region (SEQ ID NO. 21) and the amino acid sequence of the cyno32 cynomolgus monkey heavy chain constant region (SEQ ID NO. 22).

FIG. 12 shows the cDNA nucleotide sequence encoding the cyno3-18 cynomolgus monkey heavy chain constant region (SEQ ID NO. 23) and the amino acid sequence of the cyno3-18 cynomolgus monkey heavy chain constant region (SEQ ID NO. 24).

FIG. 13 shows the cDNA nucleotide sequence encoding the cyno1-3 cynomolgus monkey heavy chain constant region (SEQ ID NO. 25) and the amino acid sequence of the cyno1-3 cynomolgus monkey heavy chain constant region (SEQ ID NO. 26).

FIG. 14 shows the cDNA nucleotide sequence encoding the cyno1-4 cynomolgus monkey heavy chain constant region (SEQ ID NO. 27) and the amino acid sequence of the cyno1-4 cynomolgus monkey heavy chain constant region (SEQ ID NO. 28).

FIG. 15 shows the cDNA nucleotide sequence encoding the cynoKappa cynomo(gus monkey light chain constant region (SEQ ID NO. 29) and the amino acid sequence of the cynokappa cynomolgus monkey light chain constant region (SEQ ID NO. 30).

FIG. 16 shows nucleotide sequence alignments for certain exemplary cynologous monkey immunoglobulin constant region sequence. The constant regions can be divided into three sequence families, with the hinge encoding regions showing the most variation between families. Sequence highlighted in bold is endogenous sequence that corresponds to the primers used for cloning. A. Five constant regions with similar hinge encoding sequences. B. Five constant regions with similar hinge regions. In this case there is an insert of 21 nucleotides found in two constant regions, cyno686 and cyno439, that is not present in cyno2-4, cyno2-4cys, or cyno 2-4ds. Cyono2-4 and cyno2-4cys are identical except at nucleotide 41 where there is a G for C substitution that allows for a Cys codon rather than a Ser codon. Cyno 2-4ds1 includes the first 288 nucleotides of cyno33 replacing the first 288 nucleotides of Cyno2-4. C. Four related constant regions.

FIG. 17 shows an amino acid sequence alignment of certain cynologous monkey immunoglobulin constant regions sequences. Italic text indicates the C_{H}1 region, bold text indicates the hinge region, regular text indicates the C_{H}2 region, and italic bold text indicates the C_{H}3 region.

FIG. 18 shows certain exemplary nucleotide sequences (A) and amino acid sequences (B) that may be used as variable regions on a chimeric heavy chain. Framework (FR) and CDR regions are shown.

FIG. 19 shows certain exemplary nucleotide sequences (A) and amino acid sequences (B) that may be used as variable regions on a chimeric light chain. Framework (FR) and CDR regions are shown.

### DETAILED DESCRIPTION OF CERTAIN PREFERRED EMBODIMENTS

The section headings used herein are for organizational purposes only and are not to be construed as limiting the subject matter described. All references or portions of references cited in this application are expressly incorporated by reference herein in their entirety for any purpose.

### Definitions

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is linked to a polynucleotide which it is not linked to in nature, or (3) does not occur in nature as part of a larger sequence.

The term “isolated polypeptide” referred to herein means a polypeptide encoded by cDNA, recombinant RNA, or synthetic origin or some combination thereof, which (1) is free of at least some proteins with which it would normally be found, (2) is essentially free of other proteins from the same source, e.g., from the same species, (3) is expressed by a cell from a different species, or (4) does not occur in nature.

The term “polypeptide” is used herein as a generic term to refer to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than those normally encoded by a codon.

Polypeptides include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. Such modifications may be present to the same or varying degrees at several sites in a given polypeptide. Also, in certain embodiments, a given polypeptide may contain many types of modifications such as deletions, additions, and/or substitutions of one or more amino acids of a native sequence. In certain embodiments, polypeptides may be branched as a result of ubiquitination, and, in certain embodiments, they may be cyclic, with or without branching. Cyclic, branched and branched cyclic polypeptides may result from post-translation natural processes or may be made by synthetic methods. Modifications include, but are not limited to, acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. The term “polypeptide” also encompasses sequences that comprise the amino acid sequence of cyno3-16, cyno33, cyno2-4, cyno2-4cys, cynods1, cyno439, cyno686, cyno35, cyno36, cyno477, cyno32, cyno3-18, cyno1-3, cyno1-4, cynokappa, H1, H2, H3, H4, H5, H6, H7, H8, H9, H10, H11, H12, H13, H14, L1, L2, L3, L4, L5, L6 (as described below, SEQ ID NOS:2, 4, 6, 8, 10, 12, 14, 16, 18, 20, 22, 24, 26, 28, 30, 61-74, and 81-86), and sequences that have deletions, additions, and/or substitutions of one or more amino acid of those sequences.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to components that are in a relationship permitting them to function in their intended manner. For example, a control sequence “operably linked” to a coding sequence is ligated in such a way that expression of the coding sequence is achieved under conditions compatible with the control sequences.

The term “control sequence” as used herein refers to polynucleotide sequences which may effect the expression and processing of coding sequences to which they are ligated. The nature of such control sequences may differ depending upon the host organism. According to certain embodiments, control sequences for prokaryotes may include promoter, ribosomal binding site, and transcription termination sequence. According to certain embodiments, control sequences for eukaryotes may include promoters and transcription termination sequence. In certain embodiments, “control sequences” can include leader sequences and/or fusion partner sequences.

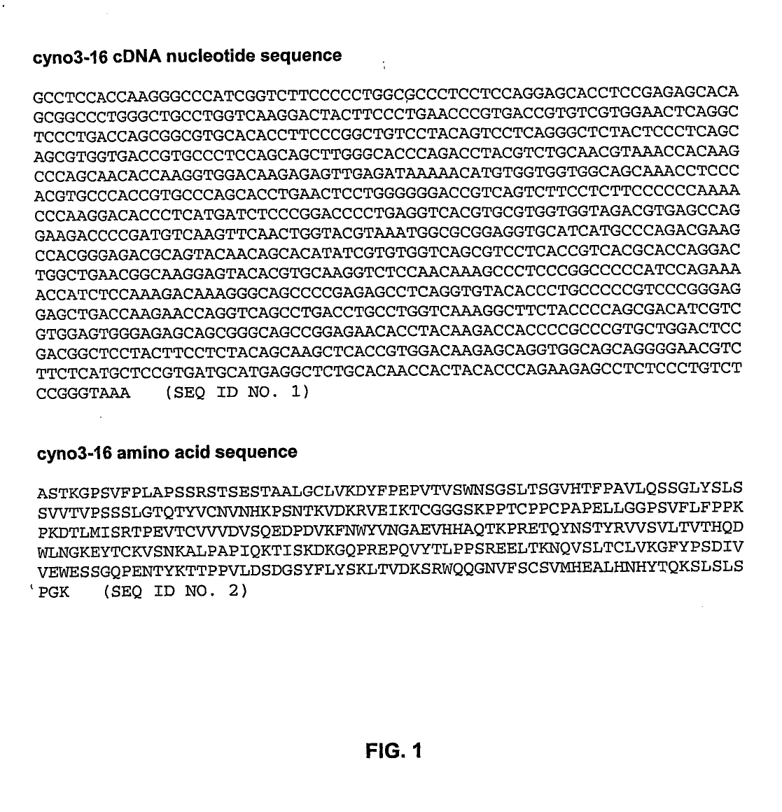

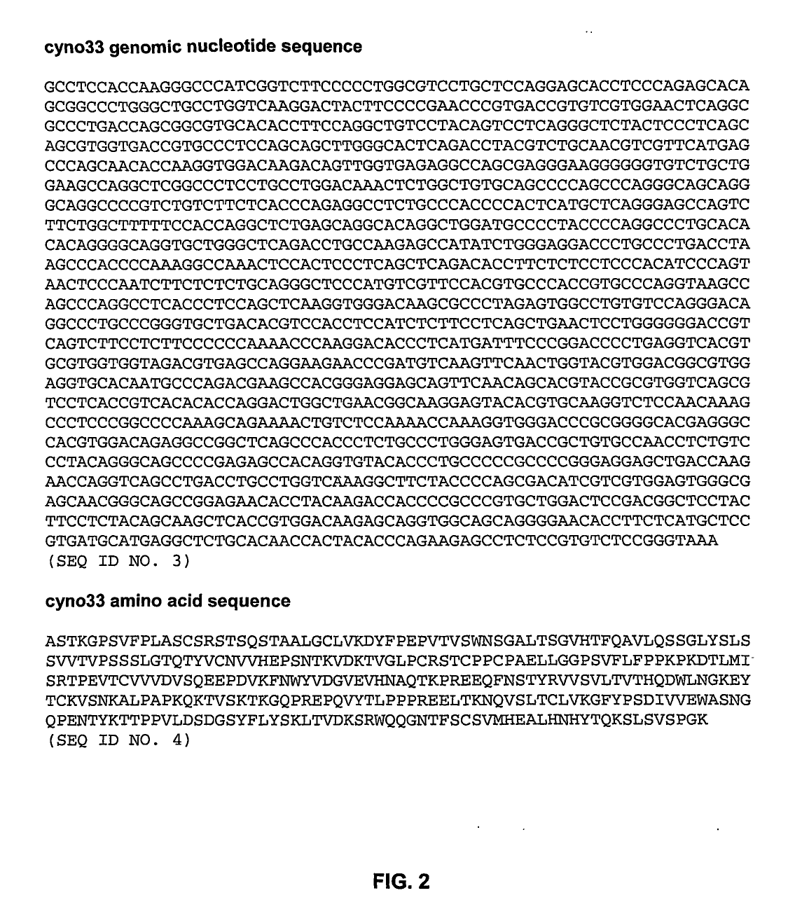

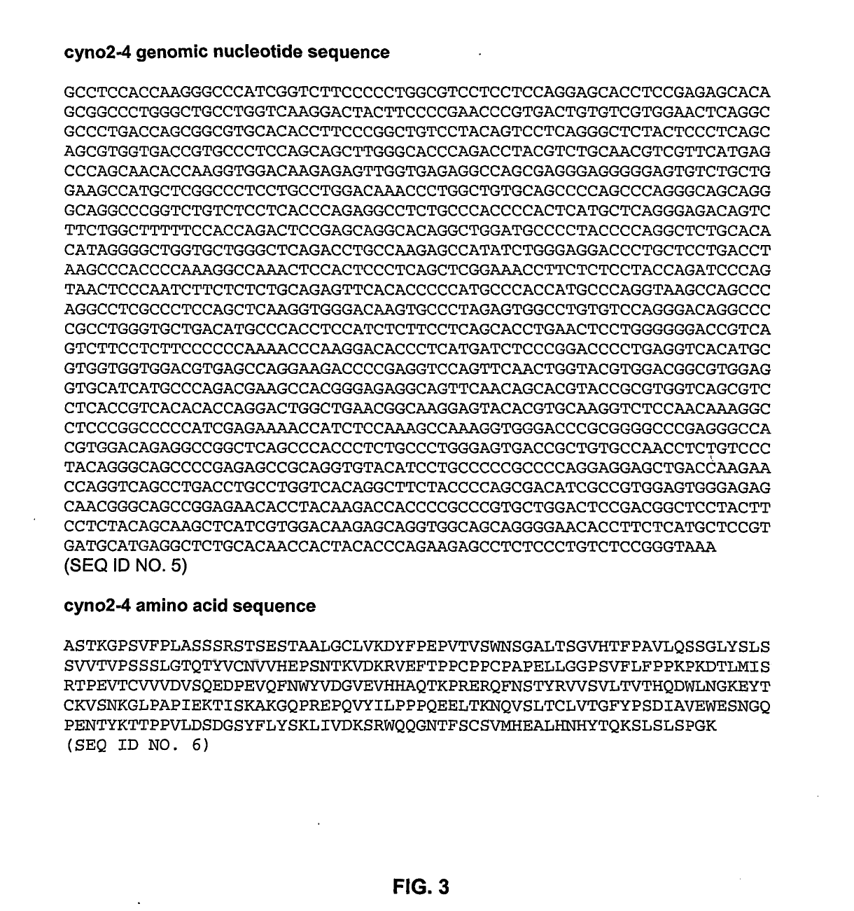

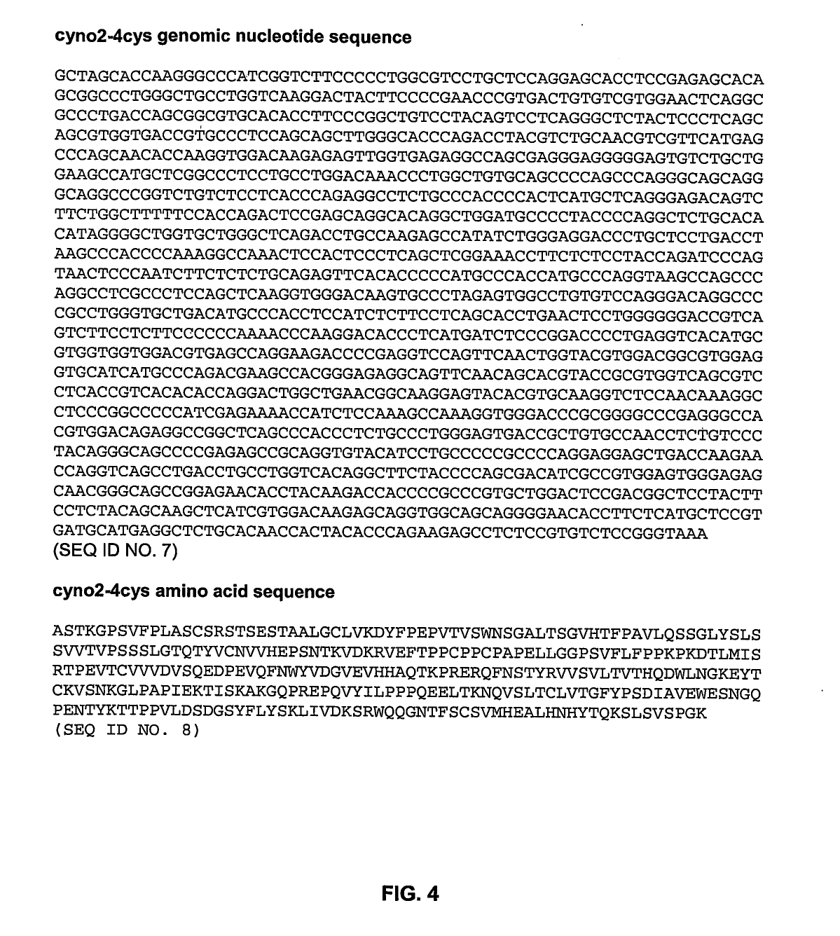

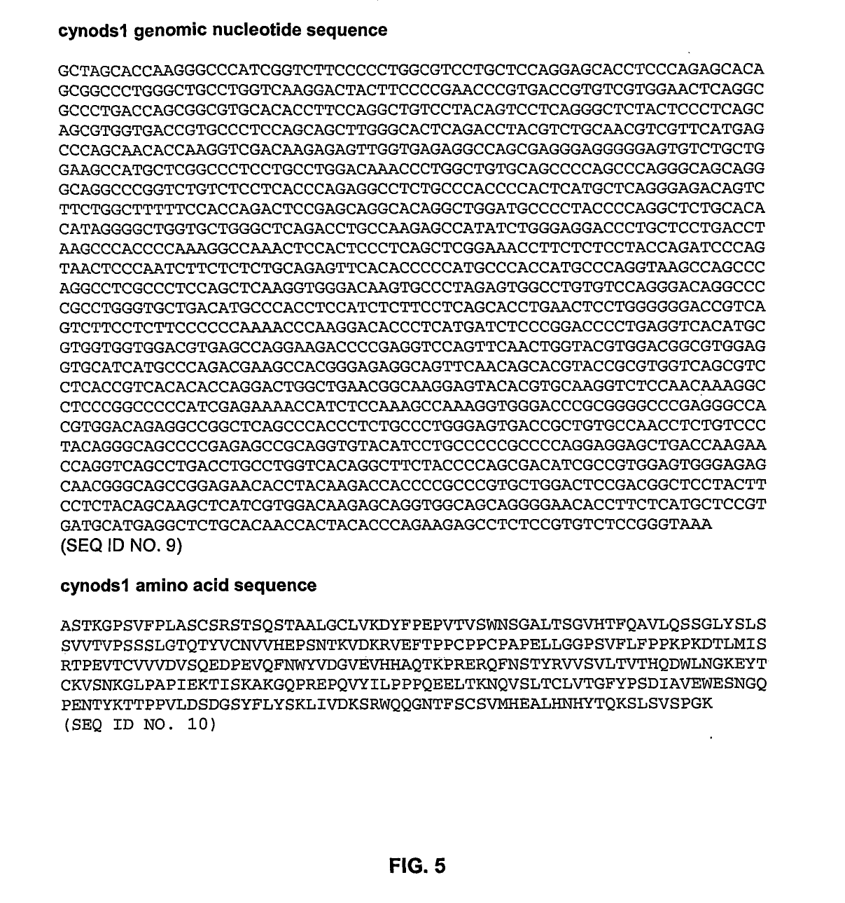

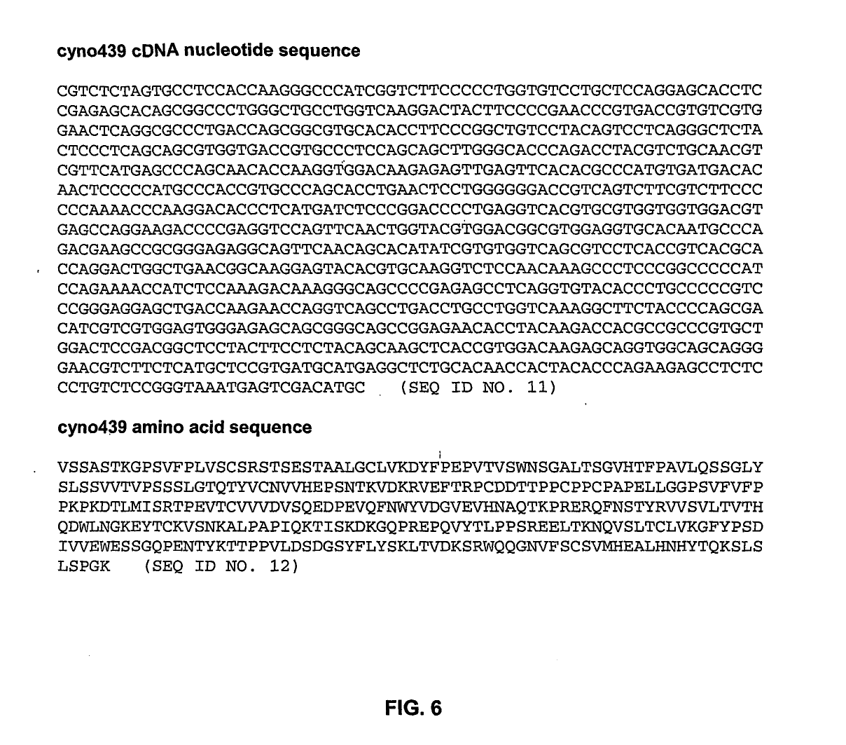

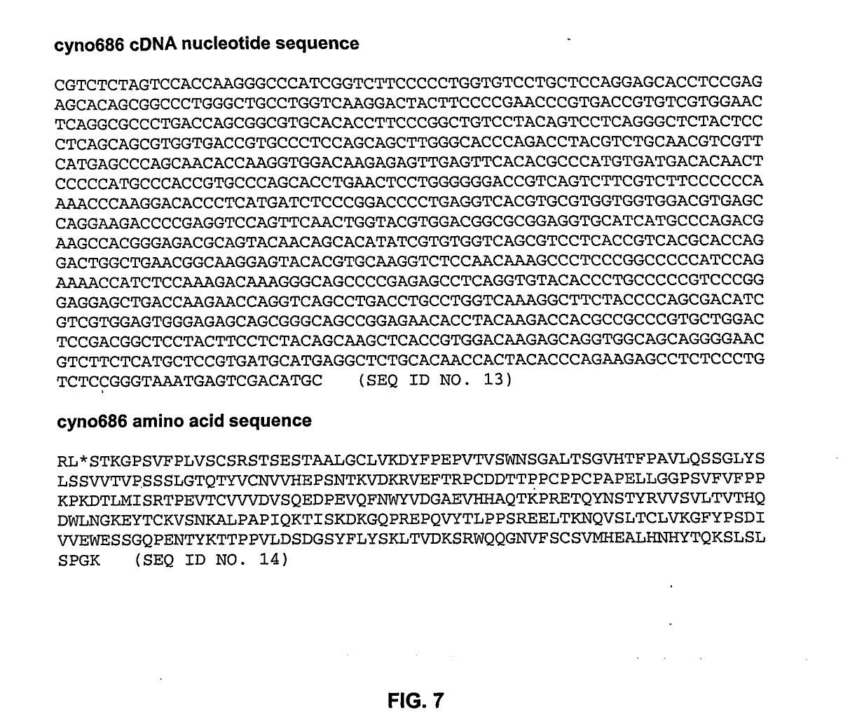

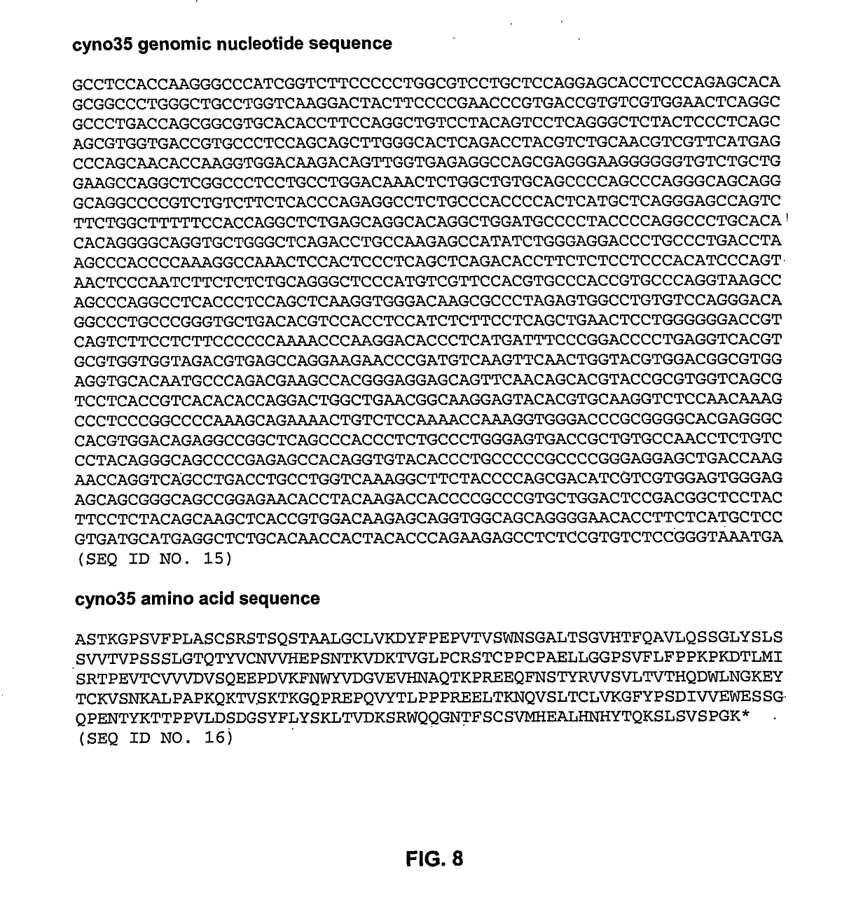

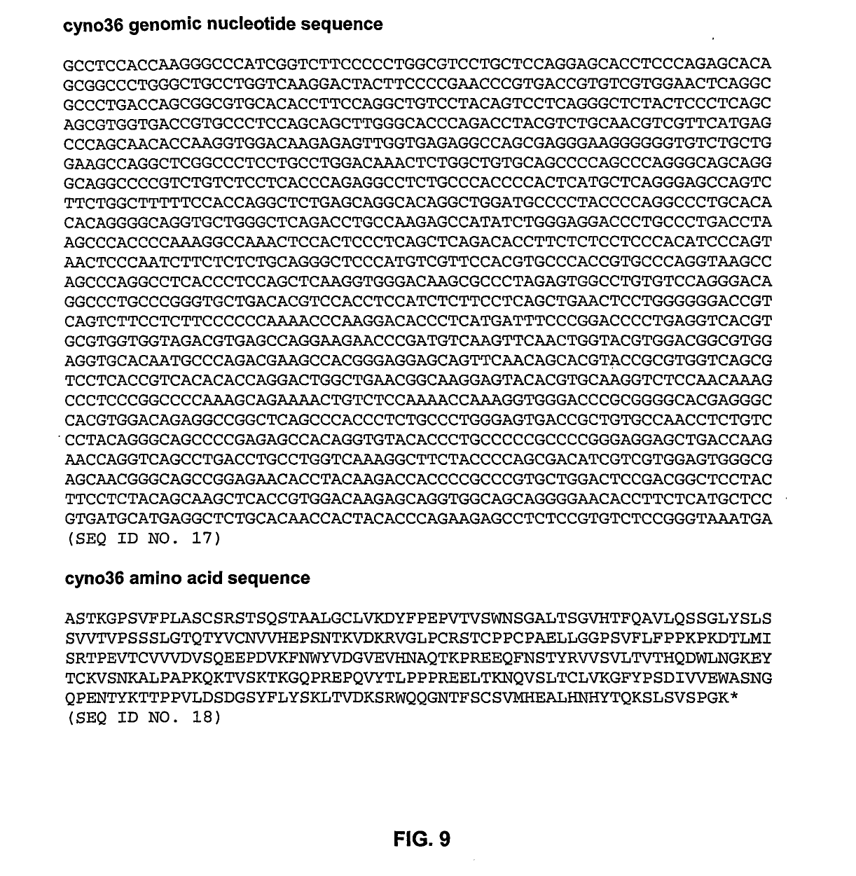

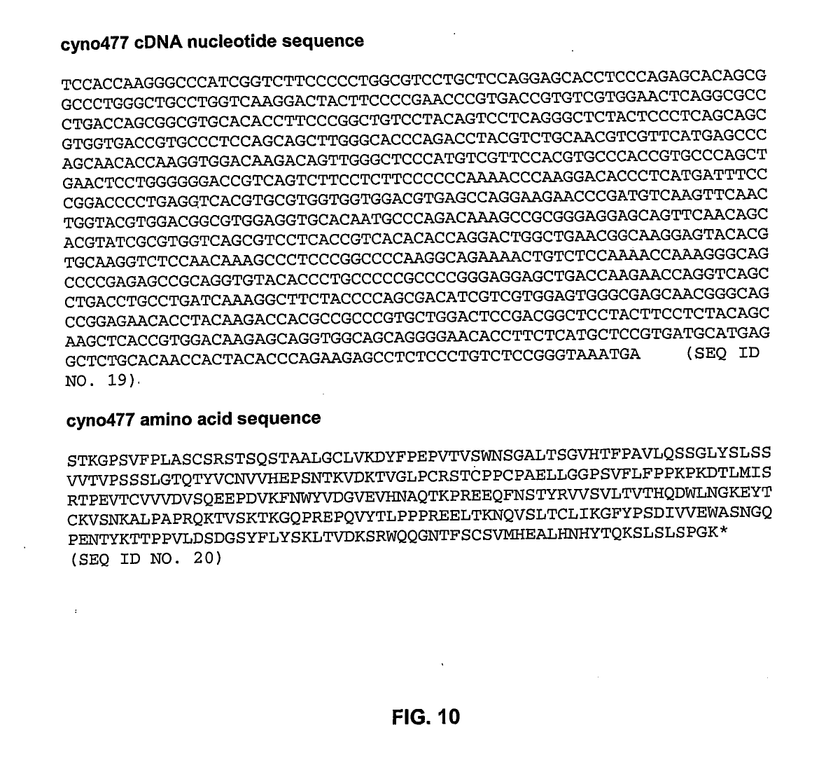

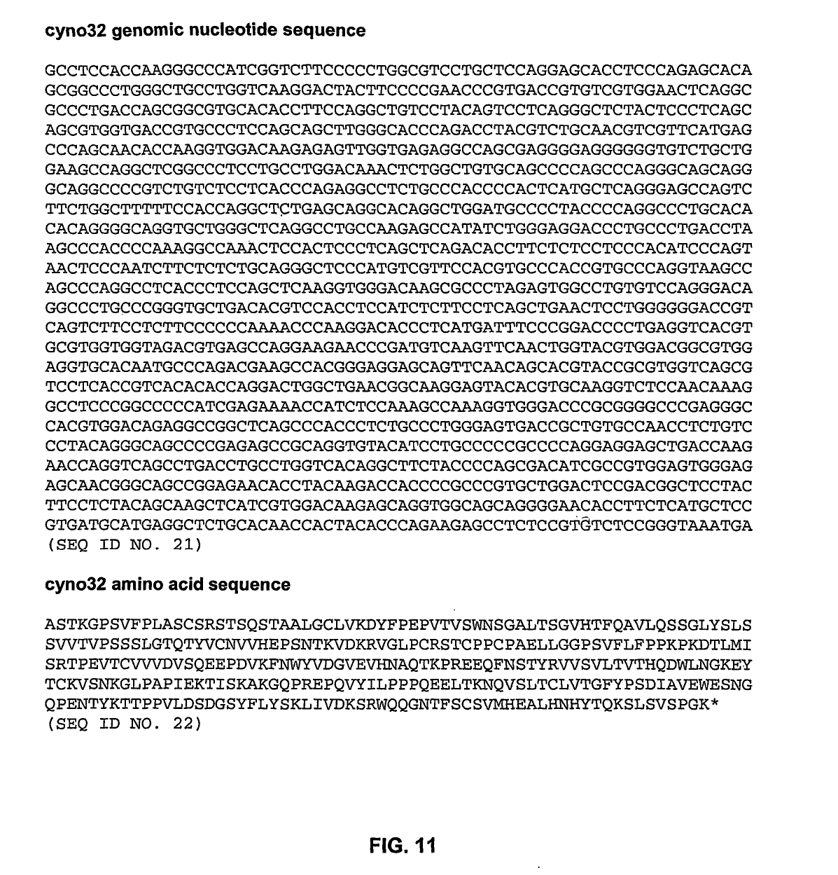

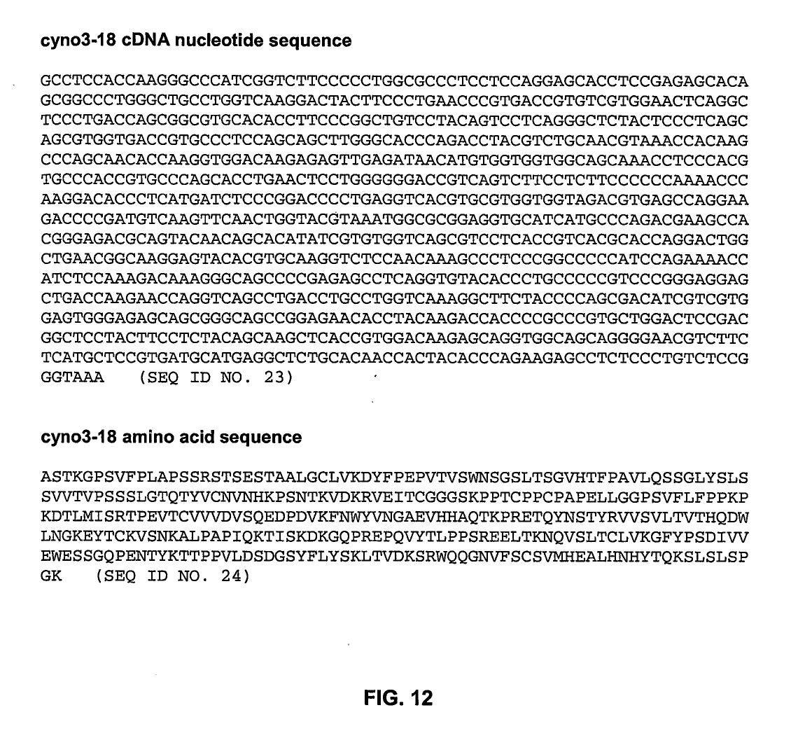

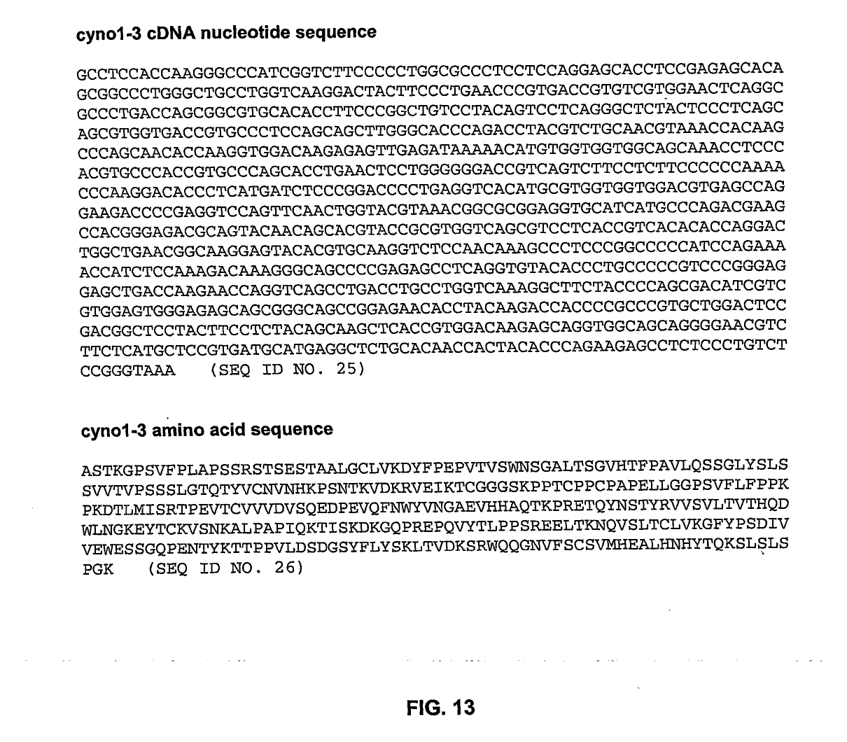

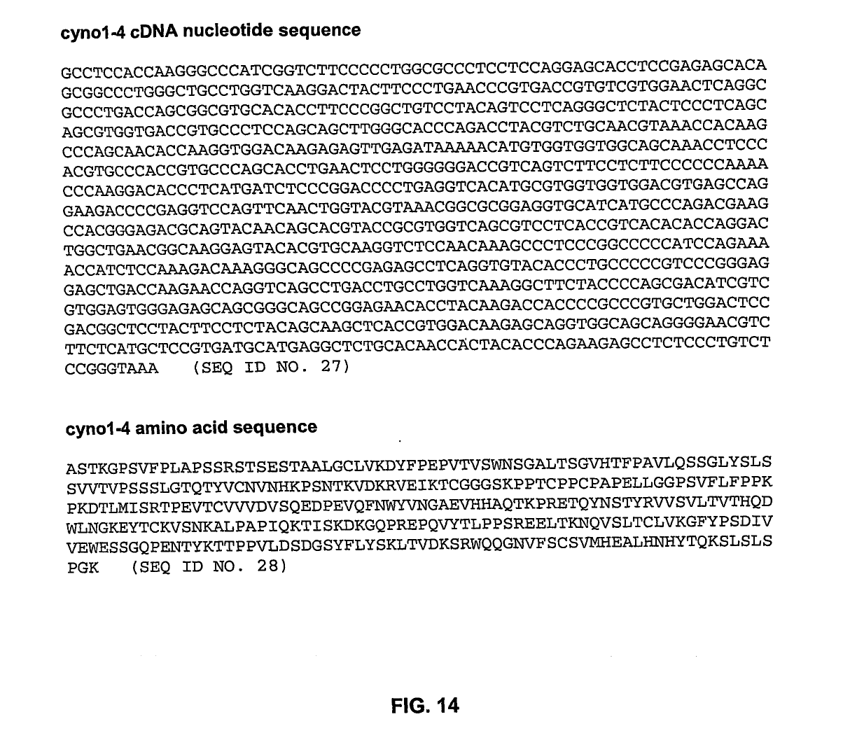

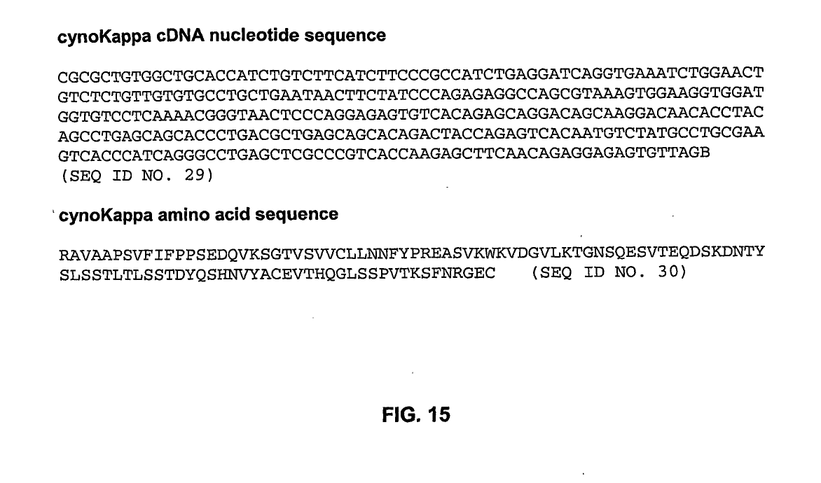

The term “polynucleotide” as referred to herein means a polymeric form of nucleotides of at least 10 bases in length. In certain embodiments, the bases may comprise at least one of ribonucleotides, deoxyribonucleotides, and a modified form of either type of nucleotide. The term includes single and double stranded forms of DNA. The term “polynucleotide” also encompases sequences that comprise SEQ ID NOS:1, 3, 5, 7, 9, 11, 13, 15, 17, 19, 21, 23, 25, 27, 29, 47-60, and 75-80. In certain embodiments, polynucleotides have nucleotide sequences that are about 90 percent, or about 95 percent, or about 96 percent, or about 97 percent, or about 98 percent, or about 99 percent identical to nucleotide sequences shown in FIGS. 1-15, 18A, and 19A.

The term “oligonucleotide” referred to herein includes naturally occurring and/or modified nucleotides linked together by naturally occurring, and/or non-naturally occurring oligonucleotide linkages. Oligonucleotides are a polynucleotide subset generally comprising a length of 200 bases or fewer. In certain embodiments, oligonucleotides are 10 to 60 bases in length. In certain embodiments, oligonucleotides are 12, 13, 14, 15, 16, 17, 18, 19, or 20 to 40 bases in length. Oligonucleotides may be single stranded or double stranded, e.g. for use in the construction of a gene mutant. Oligonucleotides of the invention may be sense or antisense oligonucleotides.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotides linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference in their entirety for any purpose. In certain instances, an oligonucleotide can include a label for detection.

Identity and similarity of related polypeptides can be readily calculated by known methods. Such methods include, but are not limited to, those described in Computational Molecular Biology, Lesk, A. M., ed., Oxford University Press, New York (1988); Biocomputing: Informatics and Genome Projects, Smith, D. W., ed., Academic Press, New York (1993); Computer Analysis of Sequence Data, Part 1, Griffin, A. M., and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., M. Stockton Press, New York (1991); and Carillo et al., SIAM J. Applied Math., 48:1073 (1988). In certain embodiments, polypeptides have amino acid sequences that are about 90 percent, or about 95 percent, or about 96 percent, or about 97 percent, or about 98 percent, or about 99 percent identical to amino acid sequences shown in FIGS. 1-15, 18B, and 19B.

Preferred methods to determine identity are designed to give the largest match between the sequences tested. Methods to determine identity are described in publicly available computer programs. Preferred computer program methods to determine identity between two sequences include, but are not limited to, the GCG program package, including GAP (Devereux et al., Nucl. Acid. Res., 12:387 (1984); Genetics Computer Group, University of Wisconsin, Madison, Wis., BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra (1990)). The well-known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full-length sequences. Accordingly, in certain embodiments, the selected alignment method (GAP program) will result in an alignment that spans at least 50 contiguous amino acids of the target polypeptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). In certain embodiments, a gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see Dayhoff et al., Atlas of Protein Sequence and Structure, 5(3) (1978) for the PAM 250 comparison matrix; Henikoff et al., Proc. Natl. Acad. Sci USA, 89:10915-10919 (1992) for the BLOSUM 62 comparison matrix) is also used by the algorithm.

In certain embodiments, the parameters for a polypeptide sequence comparison include the following:
Algorithm: Needleman et al., J. Mol. Biol. 48:443-453 (1970);
Comparison matrix: BLOSUM 62 from Henikoff et aL, supra (1992);
Gap Penalty: 12
Gap Length Penalty: 4
Threshold of Similarity: 0

The GAP program may be useful with the above parameters. In certain embodiments, the aforementioned parameters are the default parameters for polypeptide comparisons (along with no penalty for end gaps) using the GAP algorithm.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference in its entirety for any purpose. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include, but are not limited to: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences.”

Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

Naturally occurring residues may be divided into classes based on common side chain properties:
1 ) hydrophobic: norleucine, Met, Ala, Val, Leu, lie;
2) neutral hydrophilic: Cys, Ser, Thr, Asn, Gin;
3) acidic: Asp, Glu;
4) basic: His, Lys, Arg;
5) residues that influence chain orientation: Gly, Pro; and
6) aromatic: Trp, Tyr, Phe.

For example, non-conservative substitutions may involve the exchange of a member of one of these classes for a member from another class.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One may also identify epitopes from primary amino acid sequences on the basis of hydrophilicity. These regions are also referred to as “epitopic core regions.”

Exemplary amino acid substitutions are set forth in Table 1.

TABLE 1
Amino Acid Substitutions

| Original | | More specific |
|---|---|---|
| Residues | Exemplary Substitutions | exemplary Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of the polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of an antibody with respect to its three dimensional structure. In certain embodiments, one skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (Jones, D., Curr. Opin. Struct Biol., 7(3):377-87 (1997); Sippl et al., Structure, 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (See Holm, supra (1999), and Brenner, supra (1997)).

In certain embodiments, antibody variants include glycosylation variants wherein the number and/or type of glycosylation site has been altered compared to the amino acid sequences of the parent polypeptide. In certain embodiments, protein variants comprise a greater or a lesser number of N-linked glycosylation sites than the native protein. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created.

In certain embodiments, antibody variants include cysteine variants. In certain embodiments, cysteine variants have one or more cysteine residues that are deleted from or that are replaced by another amino acid (e.g., serine) as compared to the parent amino acid sequence. In certain embodiments, cysteine variants have one or more cysteine residues that are added to or that replace another amino acid (e.g., serine) as compared to the parent amino acid sequence. In certain embodiments, cysteine variants may be useful when antibodies are refolded into a biologically active conformation such as after the isolation of insoluble inclusion bodies. In certain embodiments, cysteine variants have fewer cysteine residues than the native protein. In certain embodiments, cysteine variants have more cysteine residues than the native protein. In certain embodiments, cysteine variants have an even number of cysteine residues to minimize interactions resulting from unpaired cysteines.

According to certain embodiments, amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physicochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

The term “polypeptide fragment” as used herein refers to a polypeptide that has an amino-terminal and/or carboxy-terminal deletion. In certain embodiments, fragments are at least 5 to 467 amino acids long. It will be appreciated that in certain embodiments, fragments are at least 5, 6, 8, 10, 14, 20, 50, 70, 100, 150, 200, 250, 300, 350, 400, or 450 amino acids long.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference for any purpose. Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference for any purpose); for example, by adding internal cysteine residues capable of forming intramolecular disulfide bridges which cyclize the peptide.

“Antibody” or “antibody peptide(s)” refer to an intact antibody, or a fragment thereof. In certain embodiments, the antibody fragment may be a binding fragment that competes with the intact antibody for specific binding. In certain embodiments, binding fragments are produced by recombinant DNA techniques. In certain embodiments, binding fragments are produced by enzymatic or chemical cleavage of intact antibodies. Binding fragments include, but are not limited to, Fab, Fab′, F(ab′)2, Fv, Facb, and single-chain antibodies. Non-antigen binding fragments include, but are not limited to, Fc fragments.

“Chimeric antibody” refers to an antibody that has an antibody variable region of a first species fused to another molecule, for example, an antibody constant region of another second species, such as a cynomolgus monkey. In certain embodiments, the first species may be different from the second species. In certain embodiments, the first species may be the same as the second species. In certain embodiments, chimeric antibodies are “monkeyized antibodies”, which have altered variable regions (through mutagenesis or CDR grafting) to match a portion of the known sequence of monkey variable regions. CDR grafting typically involves grafting the CDRs from an antibody with desired specificity onto the FRs of a monkey antibody, thereby replacing some or much of the non-monkey sequence with monkey sequence. Monkeyized antibodies, therefore, more closely match (in amino acid sequence) the sequence of monkey antibodies.

The term “heavy chain” includes any polypeptide having sufficient variable region sequence to confer specificity for a particular antigen. The term “light chain” includes any polypeptide having sufficient variable region sequence to confer specificity for a particular epitope. A full-length heavy chain includes a variable region domain, V_{H}, and three constant region domains, C_{H}1, C_{H}2, and C_{H}3. The V_{H} domain is at the amino-terminus of the polypeptide, and the C_{H}3 domain is at the carbody-terminus. The term “heavy chain”, as used herein, encompasses a full-length heavy chain and fragments thereof. A full-length light chain includes a variable region domain, V_{L}, and a constant region domain, C_{L}. Like the heavy chain, the variable region domain of the light chain is at the amino-terminus of the polypeptide. The term “light chain”, as used herein, encompasses a full-length light chain and fragments thereof. A Fab fragment is comprised of one light chain and the C_{H}1 and variable regions of one heavy chain. The heavy chain of a Fab molecule cannot form a disulfide bond with another heavy chain molecule. A Fab′ fragment contains one light chain and one heavy chain that contains more of the constant region, between the C_{H}1 and C_{H}2 domains, such that an interchain disulfide bond can be formed between two heavy chains to form a F(ab′)2 molecule. A Facb fragment is similar to a F(ab′)2 molecule, except the constant region in the heavy chains of the molecule extends to the end of the CH2 domain. The Fv region comprises the variable regions from both the heavy and light chains, but lacks the constant regions. Single-chain antibodies are Fv molecules in which the heavy and light chain variable regions have been connected by a flexible linker to form a single polypeptide chain which forms an antigen-binding region. Single chain antibodies are discussed in detail, e.g., in WO 88/01649 and U.S. Pat. Nos. 4,946,778 and 5,260,203. A Fc fragment contains the C_{H}2 and C_{H}3 domains of the heavy chain and contains more of the constant region, between the C_{H}1 and C_{H}2 domains, such that an interchain disulfide bond can be formed between two heavy chains.

A bivalent antibody other than a “multispecific” or “multifunctional” antibody, in certain embodiments, typically is understood to have each of its binding sites identical.

An antibody substantially inhibits adhesion of a ligand to a receptor when an excess of antibody reduces the quantity of receptor bound to the ligand by at least about 20%, 40%, 60%, 80%, 85%, or more (as measured in an in vitro competitive binding assay).

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody specifically binds an antigen when it preferentially recognizes its target antigen in a complex mixture of proteins and/or macromolecules. In certain embodiments, an antibody specifically binds an antigen when the dissociation constant is ≦1 μM, in certain embodiments, when the dissociation constant is ≦100 nM, and in certain embodiments, when the dissociation constant is ≦10 nM.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Various methods of labeling polypeptides and glycoproteins are known in the art and may be used. Examples of labels for polypeptides include, but are not limited to, the following: radioisotopes or radionuclides (e.g., 3 H, 14 C, 15 N, 35 S, 90 Y, 99 Tc, 111 In, 125 I, 131 I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

The term “biological sample”, as used herein, includes, but is not limited to, any quantity of a substance from a living thing or formerly living thing. Such living things include, but are not limited to, humans, mice, monkeys, rats., rabbits, and other animals. Such substances include, but are not limited to, blood, serum, urine, cells, organs, tissues, bone, bone marrow, lymph nodes, and skin.

The term “pharmaceutical agent or drug” as used herein refers to a chemical compound or composition capable of inducing a desired therapeutic effect when properly administered to a patient.

The term “modulator,” as used herein, is a compound that changes or alters the activity or function of a molecule. For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Certain exemplary activities and functions of a molecule include, but are not limited to, binding affinity, enzymatic activity, and signal transduction. Certain exemplary inhibitors include, but are not limited to, proteins, peptides, antibodies, peptibodies, carbohydrates or small organic molecules. Peptibodies are described, e.g., in WO01/83525.

As used herein, “substantially pure” means an object species is the predominant species present (i.e., on a molar basis it is more abundant than any other individual species in the composition). In certain embodiments, a substantially purified fraction is a composition wherein the object species comprises at least about 50 percent (on a molar basis) of all macromolecular species present. In certain embodiments, a substantially pure composition will comprise more than about 80%, 85%, 90%, 95%, or 99% of all macromolar species present in the composition. In certain embodiments, the object species is purified to essential homogeneity (contaminant species cannot be detected in the composition by conventional detection methods) wherein the composition consists essentially of a single macromolecular species.

The term “patient” includes human and animal subjects.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

In certain embodiments, this application discusses certain polynucleotides encoding heavy and light chain constant regions. In certain embodiments, this application discusses certain polypeptide sequences comprising heavy and light chain constant regions. In certain embodiments, these constant region polynucleotides and polypeptides are derived from cynomolgus monkeys. In certain embodiments a polynucleotide comprises a nucleotide sequence selected from SEQ ID NOS:1, 3, 5, 7, 9, 11, 13, 15, 17, 19, 21, 23, 25, 27, and 29. In certain embodiments, a polypeptide comprises a sequence selected from SEQ ID NOS:2, 4, 6, 8, 10, 12, 14, 16, 18, 20, 22, 24, 26, 28, and 30. In certain embodiments, a polynucleotide comprises a sequence encoding an amino acid sequence comprising a sequence selected from SEQ ID NOS:2, 4, 6, 8, 10, 12, 14, 16, 18, 20, 22, 24, 26, 28, and 30. In certain embodiments, variable region sequences corresponding to complementarity determining regions (CDR's), specifically from CDR1 through CDR3, are provided. In certain embodiments, variable region polynucleotides and polypeptides are derived from humans. In certain embodiments, the variable region polynucleotide comprises a nucleotide sequence selected from SEQ ID NOS:47-60 and SEQ ID NOS:75-80. In certain embodiments, a polypeptide comprises a sequence selected from SEQ ID NOS:61-74 and SEQ ID NOS:81-86. In certain embodiments, variable region polynucleotides and polypeptides are derived from cynomolgus monkeys. According to certain embodiments, cell lines expressing immunoglobulin molecules comprising constant regions derived from cynomolgus monkeys are also provided.

In certain embodiments, chimeric antibodies that comprise at least a portion of a monkey sequence and another species' sequence are provided. In certain embodiments, such a chimeric antibody may result in a reduced immune response in a monkey than an antibody without monkey sequences. For example, in certain instances, an antigen containing an epitope of interest may be introduced into an animal host (e.g., a mouse), thus producing antibodies specific to that epitope. In certain instances, antibodies specific to an epitope of interest may be obtained from biological samples taken from hosts that were naturally exposed to the epitope. In certain instances, introduction of human immunoglobulin (Ig) loci into mice in which the endogenous Ig genes have been inactivated offers the opportunity to obtain fully human monoclonal antibodies (MAbs). In certain instances, such antibodies from another species, may elicit an immune response to the antibodies themselves in monkeys, thus impeding evaluation of these antibodies. In certain embodiments, replacing part of the amino acid sequence of the antibody with monkey sequence may decrease the magnitude of the monkey's anti-antibody response.

In certain embodiments, a chimeric antibody comprises a variable region from a first species and a constant region from a second species. In certain embodiments, the constant region is a cynomolgus monkey constant region. Exemplary variable regions include, but are not limited to, human, mouse, pig, guinea pig, cynomolgus monkey, and rabbit variable regions. In certain embodiments, the framework regions of the variable region in the heavy chain and light chain may be replaced with framework regions derived from cynomolgus monkey sequences.

Chimeric antibodies may be produced by methods well known to those of ordinary skill in the art. In certain embodiments, the polynucleotide of the first species encoding the heavy chain variable region and the polynucleotide of the second species encoding the heavy chain constant region can be fused. In certain embodiments, the polynucleotide of the first species encoding the light chain variable region and the nucleotide sequence of the second species encoding the light chain constant region can be fused. In certain embodiments, these fused nucleotide sequences can be introduced into a cell either in a single expression vector (e.g., a plasmid). In certain embodiments, a cell comprising at least one expression vector may be used to make polypeptide. In certain embodiments, these fused nucleotide sequences can be introduced into a cell either in separate expression vectors. In certain embodiments, the host cell expresses both the chimeric heavy chain and the chimeric light chain, which combine to produce a chimeric antibody. In certain embodiments, a cell comprising at least one expression vector may be used to make a chimeric antibody. Exemplary methods for producing and expressing chimeric antibodies are discussed below.

In certain embodiments, functional domains, C_{H}1, C_{H}2, C_{H}3, and intervening sequences can be shuffled to create a different antibody constant region. For example, in certain embodiments, such hybrid constant regions can be optimized for half-life in serum, for assembly and folding of the antibody tetramer, and for improved effector function. In certain embodiments, modified antibody constant regions may also be produced by introducing single point mutations into the amino acid sequence of the constant region and testing the resulting antibody for improved qualities, e.g., those listed above.

In certain embodiments, chimeric antibodies, comprised of monkey amino acid sequences, may be used in developing treatments for human or animal diseases. Exemplary treatments include, but are not limited to, treatments for HIV, cancer, and inflammation. For example, in certain embodiments, one may develop a mouse antibody that binds to an epitope of a human pathogen, such as a virus, for which there is an monkey animal model for the human disease. In certain embodiments, to determine whether an antibody that binds to that particular epitope would be beneficial in treating the human, a chimeric antibody comprising a mouse antibody variable region and monkey antibody constant region could be evaluated for its efficacy in treating the disease in monkeys before attempting treatment in humans. Thus, in certain embodiments, a method for evaluating the effects of an antibody is provided comprising: a) introducing into a cynomolgus monkey a chimeric antibody comprising light chain and heavy chain variable regions from an antibody and light chain and heavy chain constant regions from a cynomolgus' monkey; and b) evaluating the effects of the chimeric antibody in the cynomolgus monkey. In certain embodiments, effects may be evaluated by measuring a reduction in the amount of pathogen in the monkey or by measuring a reduction in symptoms of the disease. Of course, the treatment is not limited to treatment of a disease caused by a pathogen. In certain embodiments, a disease may be established in a monkey by other methods including introduction of a substance (such as a carcinogen) and genetic manipulation. In certain embodiments, effects may be evaluated by detecting one or more adverse events in the monkey. The term “adverse event” includes, but is not limited to, an adverse reaction in a monkey that receives an antibody that is not present in a monkey that does not receive the antibody. In certain embodiments, adverse events include, but are not limited to, a fever, an immune response to an antibody, inflammation, or death of the monkey.

Naturally Occurring Antibody Structure

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that may be responsible for effector function. Antibody effector functions include activation of complement and stimulation of opsonophagocytosis. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, lgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology Ch. 7 (Paul, W., ed., 2nd ed. Raven Press, N.Y. (1989)) (incorporated by reference in its entirety for all purposes). The variable regions of each light/heavy chain pair typically form the antigen binding site.

The variable regions typically exhibit the same general structure of relatively conserved framework regions (FR) joined by three hyper variable regions, also called complementarity determining regions or CDRs. The CDRs from the two chains of each pair typically are aligned by the framework regions, which may enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk, J. Mol. Biol. 196:901-917 (1987); Chothia et al., Nature 342:878-883 (1989).

Bispecific or Bifunctional Antibodies

A bispecific or bifunctional antibody typically is an artificial hybrid antibody having two different heavy/light chain pairs and two different binding sites. Bispecific antibodies may be produced by a variety of methods including, but not limited to, fusion of hybridomas or linking of Fab′ fragments. See, e.g., Songsivilai & Lachmann, Clin. Exp. Immunol. 79:315-321 (1990), Kostelny et al. J. Immunol. 148:1547-1553 (1992).

In certain embodiments, the present invention provides a fusion protein comprising all or a functional portion of a heavy and/or a light chain cynomologus monkey antibody constant region. The fusion protein can comprise any desired additional polypeptide sequence, optionally including one or more linker sequences. The additional polypeptide sequence can comprise, for example, all or part of a naturally occurring polypeptide sequence. Any naturally occurring polypeptide sequence, or portion thereof, can be used, for example, a polypeptide sequence from a protein that binds to another molecule, e.g., to another protein. Examples of naturally-occurring polypeptide sequences that bind to another protein include sequences derived from a receptor protein, a ligand protein, a multimerizing protein, a transcription factor protein, a ribosomal protein, and a cytoskeletal protein. Other examples of naturally-occurring polypeptide sequences suitable for use in such fusion proteins include polypeptide sequences having an enzymatic activity, for example, a protein-modifying enzymatic activity, e.g., a kinase, phosphatase, or protease activity. In other embodiments, the additional polypeptide sequence is not naturally occurring. It can be, for example, a modified, mutated, or otherwise derived version of a naturally occurring protein sequence. Alternatively, it can be an artificial sequence. In one such embodiment, the non-naturally occurring polypeptide sequence confers a desired property to the fusion protein, for example, stability, solubility, detectability, or the like. In one embodiment, the non-naturally occurring polypeptide sequence allows the fusion protein to bind to a desired target molecule, for example, to another protein. Examples of target proteins include receptor proteins and ligands. The fusion protein can, for example, have no effect on the functioning of the target, or it can affect the functioning of the target, e.g., it can increase or decrease the level of function of the target molecule. The fusion protein can exert its effect on the target protein via any mechanism, for example, by sterically hindering the interaction of the target with its effector and/or substrate molecule(s), or by allosterically altering the target molecule's affinity for its effector and/or substrate molecule(s). Polypeptide sequences suitable for embodiments of the fusion proteins of the invention can be designed or selected using any technique know in the art. In one embodiment, a library of fusion proteins is made, and one or more individual fusion proteins are selected from the library by their ability to bind to a desired target molecule. Further examples of methods and compositions relating to fusion proteins of the present invention can be found in U.S. Pat. No. 6,660,843, incorporated herein by reference in its entirety. In certain embodiments, the fusion proteins of the invention are provided as part of pharmaceutical compositions suitable for use in a subject, e.g., in a primate such as a cynomologus monkey or a human. In other embodiments, the invention provides methods of treating a subject, e.g., a primate such as a cynomologus monkey or a human, using fusion protein of the invention.

Preparation of Antibodies

In certain embodiments, conservative modifications to the heavy and light chains of a chimeric cynomologus monkey antibody (and corresponding modifications to the encoding nucleotides) will produce antibodies having functional and chemical characteristics similar to those of the original chimeric antibody. In contrast, substantial modifications in the functional and/or chemical characteristics of a chimeric cynomologus monkey antibody may be accomplished by selecting substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis.”

Desired amino acid substitutions (whether conservative or non-conservative) can be determined by those skilled in the art at the time such substitutions are desired. In certain embodiments, amino acid substitutions can be used to identify important residues of the chimeric cynomologus monkey antibody, such as those which may increase or decrease the affinity of the chimeric antibodies to given antigen or the effector function of the chimeric antibodies.

In certain embodiments, antibodies can be expressed in cell lines other than hybridoma cell lines. In certain embodiments, sequences encoding particular antibodies, including chimeric antibodies, can be used for transformation of a suitable mammalian host cell. According to certain embodiments, transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference for any purpose). In certain embodiments, the transformation procedure used may depend upon the host to be transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include, but are not limited to, many immortalized cell lines available from the American Type Culture Collection (ATCC), including but not limited to Chinese hamster ovary (CHO) cells, E5 cells, HeLa cells, baby hamster kidney (BHK) cells, monkey kidney cells (COS), human hepatocellular carcinoma cells (e.g., Hep G2), and a number of other cell lines. In certain embodiments, cell lines may be selected through determining which cell lines have high expression levels and produce antibodies with constitutive antigen binding properties.

According to certain embodiments, antibodies are useful for detecting a particular antigen in biological samples. In certain embodiments, this allows the identification of cells or tissues which produce the protein. In certain embodiments, antibodies which bind to a particular protein and block interaction with other binding compounds may have therapeutic use.

In certain embodiments, methods are provided of treating a patient comprising administering a therapeutically effective amount of an antibody. In certain such embodiments, the additional therapeutic agent is administered in a therapeutically effective amount.

In certain embodiments, an antibody is used in conjunction with a therapeutically effective amount of an additional therapeutic agent. Exemplary therapeutic agents include, but are not limited to, the bone morphogenic factors designated BMP-1 through BMP-12; transforming growth factor-β (TGF-β) and TGF-β family members; interleukin-1 (IL-1) inhibitors, including, but not limited to, IL-1ra and derivatives thereof and Kinere™; TNFα inhibitors, including, but not limited to, soluble TNFα receptors, Enbrel™, anti-TNFα antibodies, Remicade™, and D2E7 antibodies; parathyroid hormone and analogs thereof; parathyroid related protein and analogs thereof; E series prostaglandins; bisphosphonates (such as alendronate and others); bone-enhancing minerals such as fluoride and calcium; non-steroidal anti-inflammatory drugs (NSAIDs), including, but not limited to, COX-2 inhibitors, such as Celebrex™ and Vioxx™; immunosuppressants, such as methotrexate or leflunomide; serine protease inhibitors, including, but not limited to, secretory leukocyte protease inhibitor (SLPI); IL-6 inhibitors (including, but not limited to, antibodies to IL-6), IL-8 inhibitors (including, but not limited to, antibodies to IL-8); IL-18 inhibitors (including, but not limited to, IL-18 binding protein and IL-18 antibodies); Interleukin-1 converting enzyme (ICE) modulators; fibroblast growth factors FGF-1 to FGF-10 and FGF modulators; PAF antagonists; keratinocyte growth factor (KGF), KGF-related molecules, and KGF modulators; matrix metalloproteinase (MMP) modulators; Nitric oxide synthase (NOS) modulators, including, but not limited to, modulators of inducible NOS; modulators of glucocorticoid receptor; modulators of glutamate receptor; modulators of lipopolysaccharide (LPS) levels; and noradrenaline and modulators and mimetics thereof. See, e.g., Published PCT Application No. WO 03/0002713 for exemplary details on exemplary additional therapeutic agents.

In certain embodiments, in view of the condition and the desired level of treatment, two, three, or more agents may be administered. In certain embodiments, such agents may be provided together by inclusion in the same formulation. In certain embodiments, such agents and an antibody may be provided together by inclusion in the same formulation. In certain embodiments, such agents may be provided together by inclusion in a treatment kit. In certain embodiments, such agents and an antibody may be provided together by inclusion in a treatment kit. In certain embodiments, such agents may be provided separately. In certain embodiments, when administered by gene therapy, the genes encoding protein agents and/or an antibody may be included in the same vector. In certain embodiments, the genes encoding protein agents and/or an antibody may be under the control of the same promoter region. In certain embodiments, the genes encoding protein agents and/or an antibody may be in separate vectors.

In certain embodiments, the invention provides for pharmaceutical compositions comprising a therapeutically effective amount of an antibody together with a pharmaceutically acceptable diluent, carrier, solubilizer, emulsifier, preservative and/or adjuvant.

In certain embodiments, the invention provides for pharmaceutical compositions comprising a therapeutically effective amount of an antibody and a therapeutically effective amount of at least one additional therapeutic agents, together with a pharmaceutically acceptable diluent, carrier, solubilizer, emulsifier, preservative and/or adjuvant. In certain embodiments, the at least one additional therapeutic agent is selected from bone morphogenic factors designated BMP-1 through BMP-12; transforming growth factor-β (TGF-β) and TGF-β family members; interleukin-1 (IL-1) inhibitors, including, but not limited to, IL-1ra and derivatives thereof and Kineret™; TNFα inhibitors, including, but not limited to, a soluble TNFα receptor, Enbrel™, anti-TNFα antibodies, Remicade™, and D2E7 antibody; parathyroid hormone and analogs thereof, parathyroid related protein and analogs thereof; E series prostaglandins; bisphosphonates (such as alendronate and others); fluoride and calcium; non-steroidal anti-inflammatory drugs (NSAIDs), including COX-2 inhibitors, such as Celebrex™ and Vioxx™; immunosuppressants, such as methotrexate or leflunomide; serine protease inhibitors such as secretory leukocyte protease inhibitor (SLPI); IL-6 inhibitors (e.g., antibodies to IL-6), IL-8 inhibitors (e.g., antibodies to IL-8); IL-18 inhibitors (e.g., IL-18 binding protein or IL-18 antibodies); Interleukin-1 converting enzyme (ICE) modulators; fibroblast growth factors FGF-1 to FGF-10 and FGF modulators; PAF antagonists; keratinocyte growth factor (KGF), KGF-related molecules, or KGF modulators; matrix metalloproteinase (MMP) modulators; Nitric oxide synthase (NOS) modulators, including modulators of inducible NOS; modulators of glucocorticoid receptor; modulators of glutamate receptor; modulators of lipopolysaccharide (LPS) levels; and noradrenaline and modulators and mimetics thereof. See, e.g., Published PCT Application No. WO 03/0002713 for exemplary details on exemplary additional therapeutic agents.

In certain embodiments, acceptable formulation materials preferably are nontoxic to recipients at the dosages and concentrations employed.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company (1990).

In certain embodiments, an antibody and/or an additional therapeutic molecule is linked to a half-life extending vehicle known in the art. Such vehicles include, but are not limited to, the Fc domain, polyethylene glycol, and dextran. Such vehicles are described, e.g., in U.S. application Ser. No. 09/428,082 and published PCT Application No. WO 99/25044, which are hereby incorporated by reference for any purpose.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, the primary vehicle or carrier in a pharmaceutical composition may be either aqueous or non-aqueous in nature. For example, in certain embodiments, a suitable vehicle or carrier may be water for injection, physiological saline solution or artificial cerebrospinal fluid, possibly supplemented with other materials common in compositions for parenteral administration. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which may further include sorbitol or a suitable substitute therefor. Additional pharmaceutical carriers include, but are not limited to, oils, including petroleum oil, animal oil, vegetable oil, peanut oil, soybean oil, mineral oil, sesame oil, and the like. Aqueous dextrose and glycerol solutions can also be employed as liquid carriers, particularly for injectable solutions. In certain embodiments, a composition comprising an antibody, with or without at least one additional therapeutic agents, may be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an antibody, with or without at least one additional therapeutic agents, may be formulated as a lyophilizate using appropriate excipients such as sucrose.

In certain embodiments, pharmaceutical compositions can be selected for parenteral delivery. In certain embodiments, the compositions may be selected for inhalation or for delivery through the digestive tract, such as orally. The preparation of such pharmaceutically acceptable compositions is within the skill of the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition may be in the form of a pyrogen-free, parenterally acceptable aqueous solution comprising the desired antibody, with or without additional therapeutic agents, in a pharmaceutically acceptable vehicle. In certain embodiments, a vehicle for parenteral injection is sterile distilled water in which the antibody, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide for the controlled or sustained release of the product which may then be delivered via a depot injection. In certain embodiments, hyaluronic acid may also be used, and may have the effect of promoting sustained duration in the circulation. In certain embodiments, implantable drug delivery devices may be used to introduce the desired molecule.

In certain embodiments, a pharmaceutical composition may be formulated for inhalation. In certain embodiments, an antibody, with or without at least one additional therapeutic agent, may be formulated as a dry powder for inhalation. In certain embodiments, an inhalation solution comprising an antibody, with or without at least one additional therapeutic agent, may be formulated with a propellant for aerosol delivery. In certain embodiments, solutions may be nebulized. Pulmonary administration is further described in PCT application no. PCT/US94/001875, which describes pulmonary delivery of chemically modified proteins.

In certain embodiments, it is contemplated that formulations may be administered orally. In certain embodiments, an antibody, with or without at least one additional therapeutic agents, that is administered in this fashion may be formulated with or without those carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. In certain embodiments, at least one additional agent can be included to facilitate absorption of the antibody and/or any additional therapeutic agents. In certain embodiments, diluents, flavorings, low melting point waxes, vegetable oils, lubricants, suspending agents, tablet disintegrating agents, and binders may also be employed.

In certain embodiments, a pharmaceutical composition may involve an effective quantity of antibodies, with or without at least one additional therapeutic agents, in a mixture with non-toxic excipients which are suitable for the manufacture of tablets. In certain embodiments, by dissolving the tablets in sterile water, or another appropriate vehicle, solutions may be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving antibodies, with or without at least one additional therapeutic agents, in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93100829 which describes the controlled release of porous polymeric microparticles for the delivery of pharmaceutical compositions. In certain embodiments, sustained-release preparations may include semipermeable polymer matrices in the form of shaped articles, e.g. films, or microcapsules. Sustained release matrices may include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions may also include liposomes, which can be prepared by any of several methods known in the art. See e.g., Eppstein et al., Proc. Natl. Acad. Sci, USA, 82:3688-3692 (1985); EP 036,676; EP 088,046 and EP 143,949.

In certain embodiments, the pharmaceutical composition to be used for in vivo administration is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, after the pharmaceutical composition has been formulated, it may be stored in sterile vials as a solution, suspension, gel, emulsion, solid, or as a dehydrated or lyophilized powder. In certain embodiments, such formulations may be stored either in a ready-to-use form or in a form (e.g., lyophilized) that is reconstituted prior to administration.

In certain embodiments, the present invention is directed to kits for producing a single-dose administration unit. In certain embodiments, the kits may each contain both a first container having a dried protein and a second container having an aqueous formulation. In certain embodiments of this invention, kits containing single and multi-chambered pre-filled syringes (e.g., liquid syringes and lyosyringes) are included.

In certain embodiments, the effective amount of a pharmaceutical composition comprising an antibody, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the antibody, with or without at least one additional therapeutic agent, is being used, the route of administration, and the size (body weight, body surface or organ size) and/or condition (the age and general health) of the patient. In certain embodiments, the clinician may titer the dosage and modify the route of administration to obtain the optimal therapeutic effect. In certain embodiments, a typical dosage may range from about 0.1 μg/kg to up to about 100 mg/kg or more, depending on the factors mentioned above. In certain embodiments, the dosage may range from 0.1 μg/kg up to about 100 mg/kg; or 1 μg/kg up to about 100 mg/kg; or 5 μg/kg up to about 100 mg/kg.

In certain embodiments, the frequency of dosing will take into account the pharmacokinetic parameters of the antibody and/or any additional therapeutic agents in the formulation used. In certain embodiments, a clinician will administer the composition until a dosage is reached that achieves the desired effect. In certain embodiments, the composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages may be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

In certain embodiments, the composition may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

In certain embodiments, it may be desirable to use a pharmaceutical composition comprising an antibody, with or without at least one additional therapeutic agent, in an ex vivo manner. In such instances, cells, tissues and/or organs that have been removed from the patient are exposed to a pharmaceutical composition comprising an antibody, with or without at least one additional therapeutic agent, after which the cells, tissues and/or organs are subsequently implanted back into the patient.

In certain embodiments, an antibody and/or any additional therapeutic agents can be delivered by implanting certain cells that have been genetically engineered, using methods such as those described herein, to express and secrete the polypeptides. In certain embodiments, such cells may be animal or human cells, and may be autologous, heterologous, or xenogeneic. In certain embodiments, the cells may be immortalized. In certain embodiments, in order to decrease the chance of an immunological response, the cells may be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### EXAMPLES

### Example 1

### Cloning the Heavy Chain Constant Region from Cynomolgus Monkey Sequences

Polynucleotides encoding native cynomolgus monkey antibody heavy chain constant regions are cloned as follows.

A. For the cyno3-16 constant region, RNA is isolated from cynomolgus monkey B cells purified from whole blood of a cynomolgus monkey. cDNA is synthesized from the RNA and the cDNA is used as a template for PCR with the following primers: 5-′GCCTCCACCAAGGGCCCTCG-3′ (SEQ ID NO:31) and 5′-TTTACCCGGAGACAGGGAGAG-3′ (SEQ ID NO: 32). PCR is performed using an Expand High Fidelity PCR System (Roche) with the addition of 5% DMSO. Samples are first incubated for 2 minutes at 94° C., followed by 40 cycles under the following conditions per cycle: 30 sec at 94° C; 30 seconds at either 45° C. or 50° C.; and 1 minute or 1.5 minutes at 72° C. Samples are then incubated for 7 minutes at 72° C. following the last PCR cycle. PCR primers are used at a concentration of 30 pmol and 2 ul cDNA preparation is used.

B. For the cyno2-4 and cyno33 constant regions, genomic DNA isolated from a cynomolgus monkey B cell cell line is used as a template for PCR. Two different primer sets are used for amplification of these cyno IgG constant regions. 5′-GCCTCCACCMGGGCCCTCG-3′ (SEQ ID NO:33) and 5′-TTTACCCGGAGACAGGGAGAG-3′ (SEQ ID NO:34) are used for cyno2-4 while 5′-GTCACATGGCACCACCTCTCT-3′ (SEQ ID NO:35) and 5′-GGTACGTGCCMGCATCCTCG-3′(SEQ ID NO:36) are used for cyno33. PCR reactions are performed as described in Example 1A above except that 1 μl of genomic DNA is used as a template.

Following the initial cloning, each of the polynucleotides encoding the cynomolgus monkey constant regions is constructed as an NheI-NotI cassette by introducing a NheI or NotI enzyme restriction site into the appropriate PCR primer. Specifically, nucleotide modifications are made at the 5′ end of each constant region to introduce an NheI site. This does not alter the amino acid sequence. A NotI site is introduced immediately 3′ to the termination codon.

C. The cyno2-4cys constant region is constructed by PCR site directed mutagenesis of the polynucleotide encoding the cyno2-4 sequence. Site directed mutagenesis is carried out using a QuikChange Site-Directed Mutagenesis Kit (Stratagene). The third serine in the C_{H}1 domain is converted to a cysteine by introducing a single point mutation. The primers used are: 5′-CTGGCGTCCTGCTCCAGGAGC-3′ (SEQ ID NO:37) and 5′-GCTCCTGGAGCAGGACGCCAG-3′ (SEQ ID NO:38).

D. The cynods1 constant region comprises sequences of the cyno33 and cyno2-4 constant regions. Polynucleotides encoding amino acids 1 through 94 of the C_{H}1 domain of cyno33 are amplified by PCR as described above in Example 1A, generating a NheI-SalI cassette by introducing NheI and SalI restriction sites into the PCR primers by methods known in the art. The primers used are 5′-GCTAGCACCMGGGCCCATCGGTCTT-3′ (SEQ ID NO:39) and 5′-MCTGTCTTGTCGACCTTGGTGTTG-3′ (SEQ ID NO:40). The 3′ end of the polynucleotides encoding C_{H}1, hinge, C_{H}2 and C_{H}3 domains of cyno2-4 are amplified by PCR as described above in Example 1A to generate a SalI-NotI cassette using primers 5′-CMCACCAAGGTCGACMGAGAGTT-3′ (SEQ ID NO:41) and 5′-GCGGCCGCTCATTTACCCGGAGACACGGAG-3′ (SEQ ID NO:42). Introduction of the SalI site does not alter the polypeptide sequence. The NheI-SalI cassette and the SalI-NotI cassette are ligated together to make a polynucleotide sequence encoding the cynods1 constant region. The resulting construct contains the C_{H}1 domain of cyno33 with the exception of a T to R switch at the second to last amino acid of the C_{H}1 domain. The hinge, C_{H}2 and C_{H}3 domains are encoded by the cyno2-4 polynucleotide sequence.

E. For the cyno686 and cyno439 constant regions, a cDNA library is prepared from RNA isolated from mixed cynomolgus monkey lymphoid tissues. This cDNA is used as a template for PCR, which is carried out using two primers 5′-CGTCTCTAGTGCCTCCACCMGGGCCCATC-3′ (SEQ ID NO: 43) and 5′-GCATGTCGACTCATTTACCCGGAGACAGGGAGAG-3′ (SEQ ID NO:44). The PCR reaction mixture included two microliters of each primer, the primers at a concentration of 5 picomoles per microliter; 5 microliters of Stratagene 10× Pfu buffer; a 0.5 microliter of 10 millimolar dNTPs (A, C, G, T) mix; 0.5 microliters of two and a half units per microliter Stratagene Pfu polymerase; 1 microliter of cDNA template; and 39 microliters of sterile water. The final volume of the reaction is 50 microliters. Twenty eight PCR cycles are carried using the following parameters per cycle: 20 seconds at 94° C., 30 seconds at 60° C., and 150 seconds at 74° C. The PCR products are cloned using the Invitrogen PCRII TOPO-TA cloning system (K4600-01SC) using the instructions provided by the system.

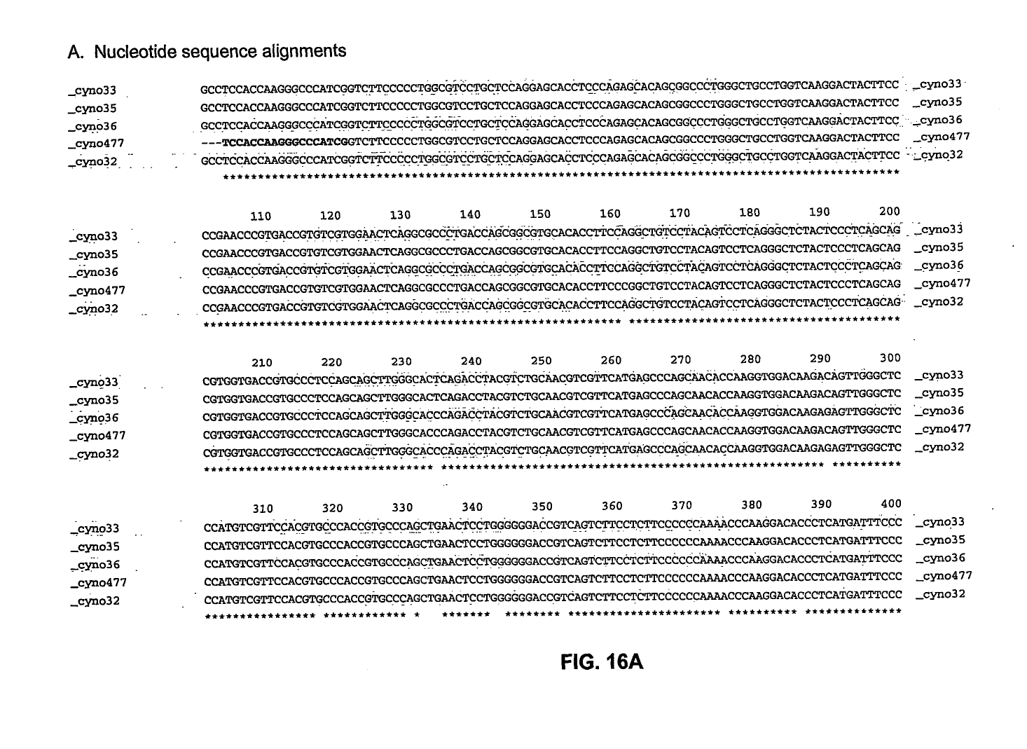

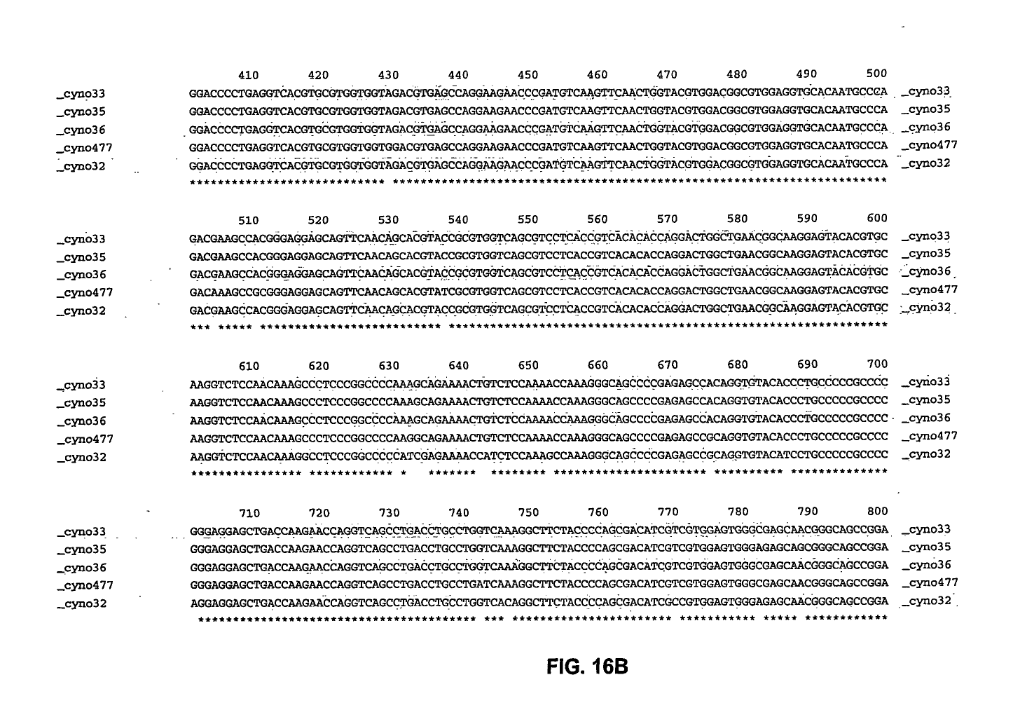

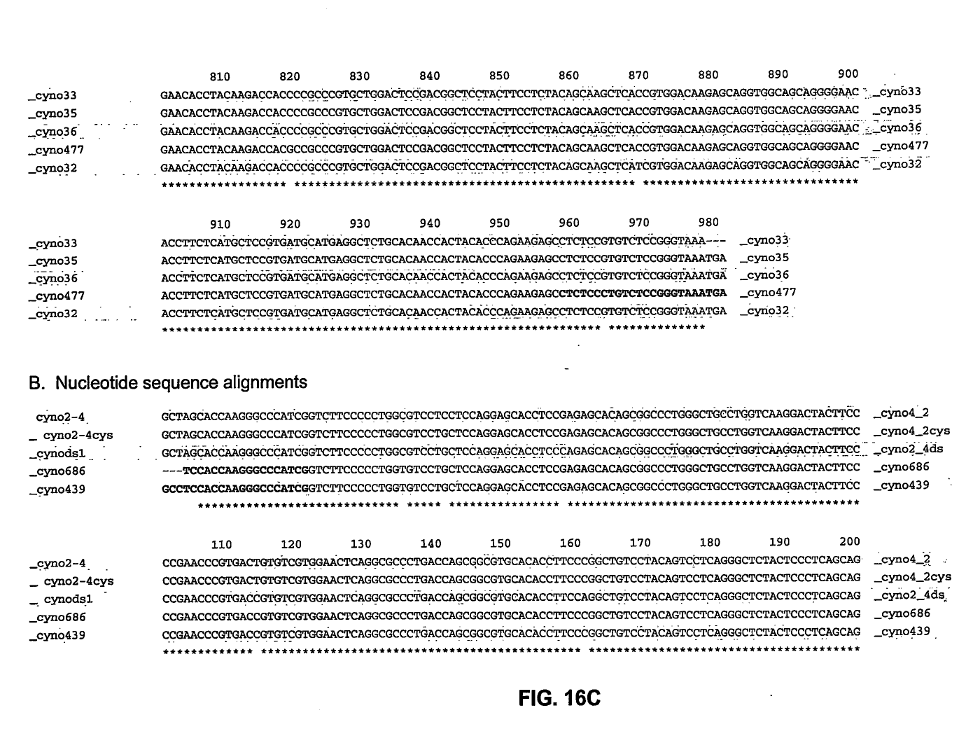

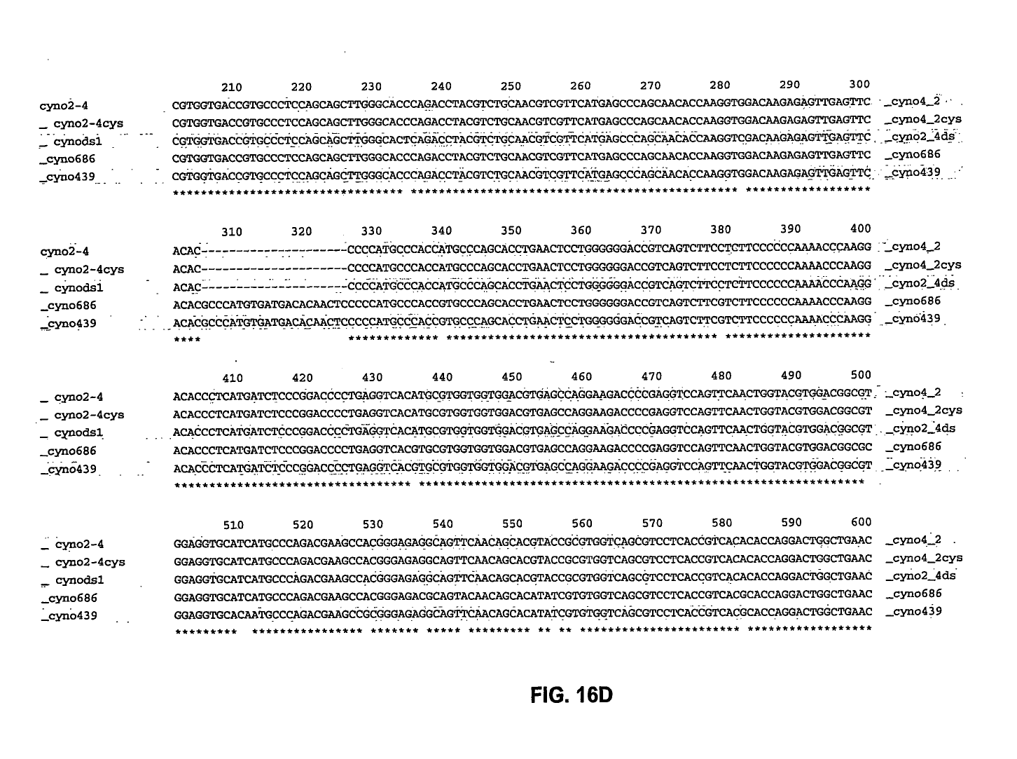

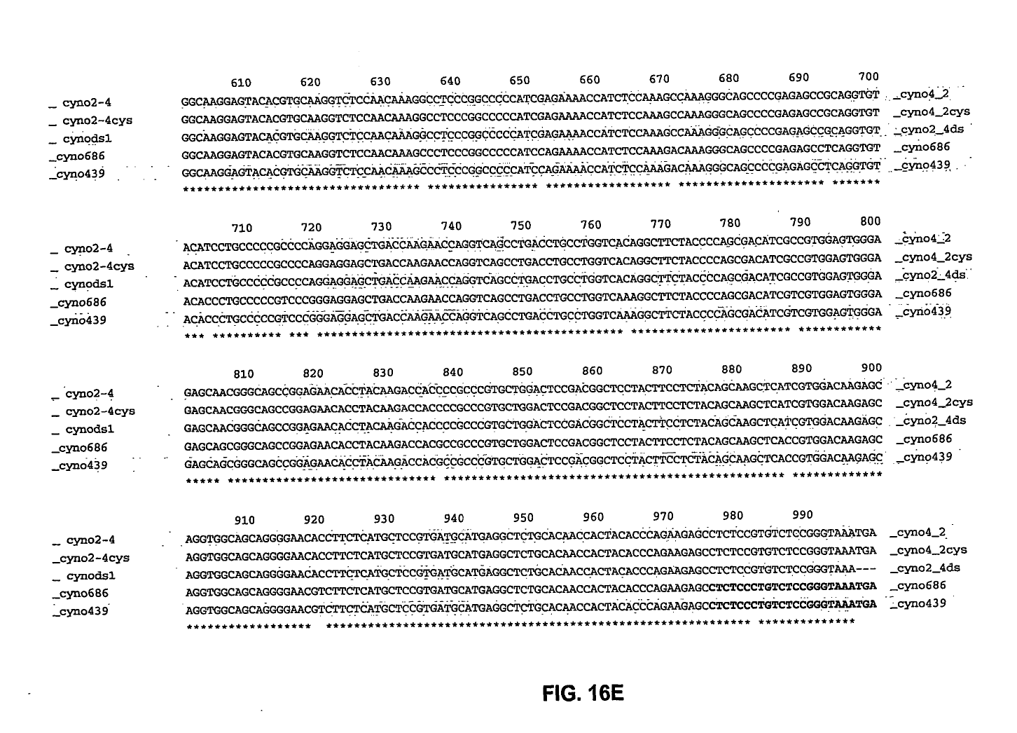

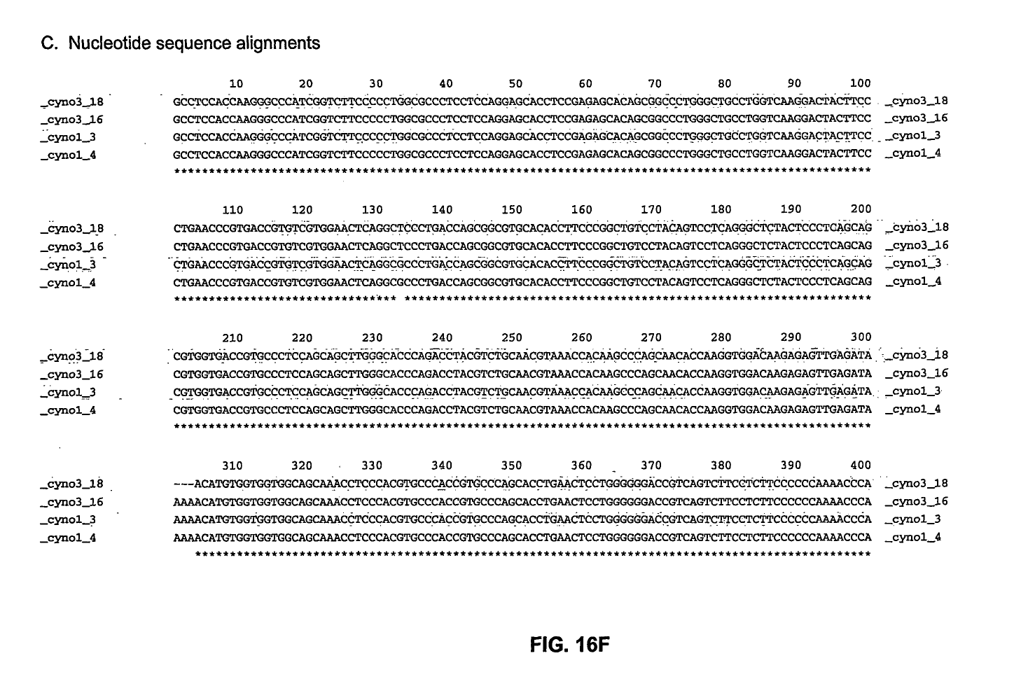

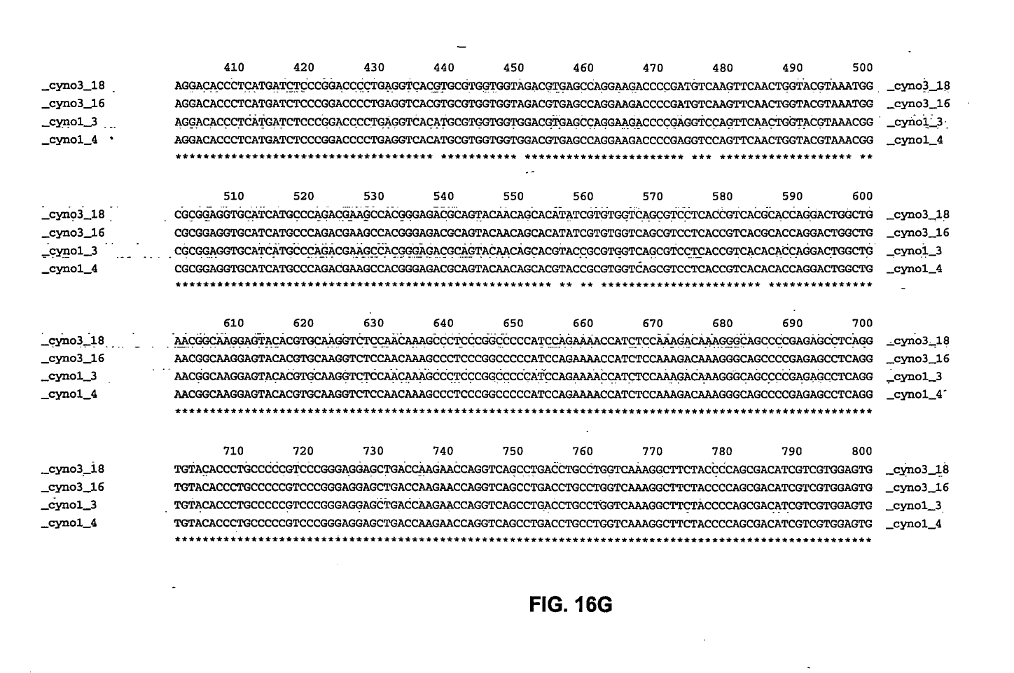

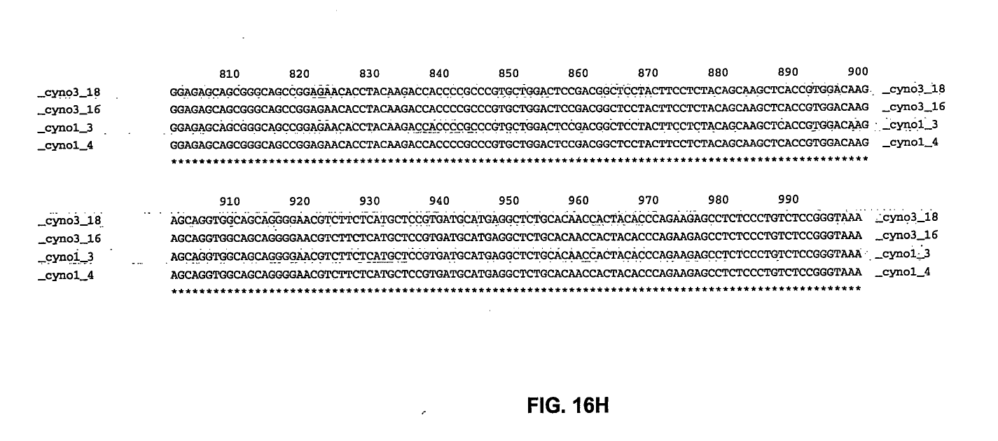

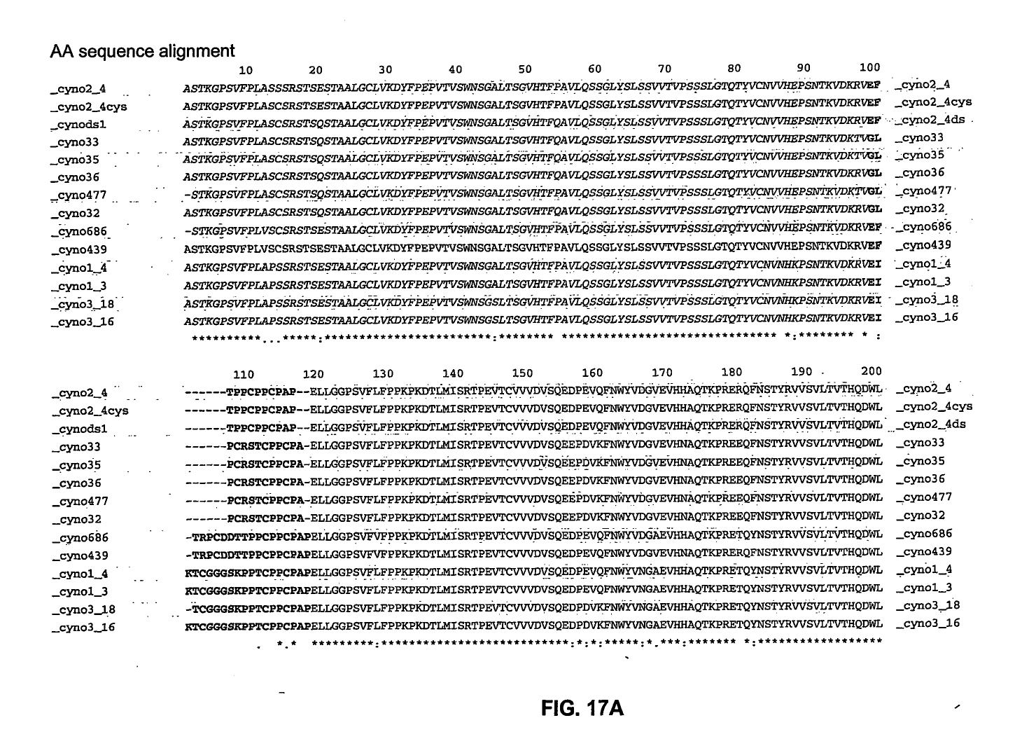

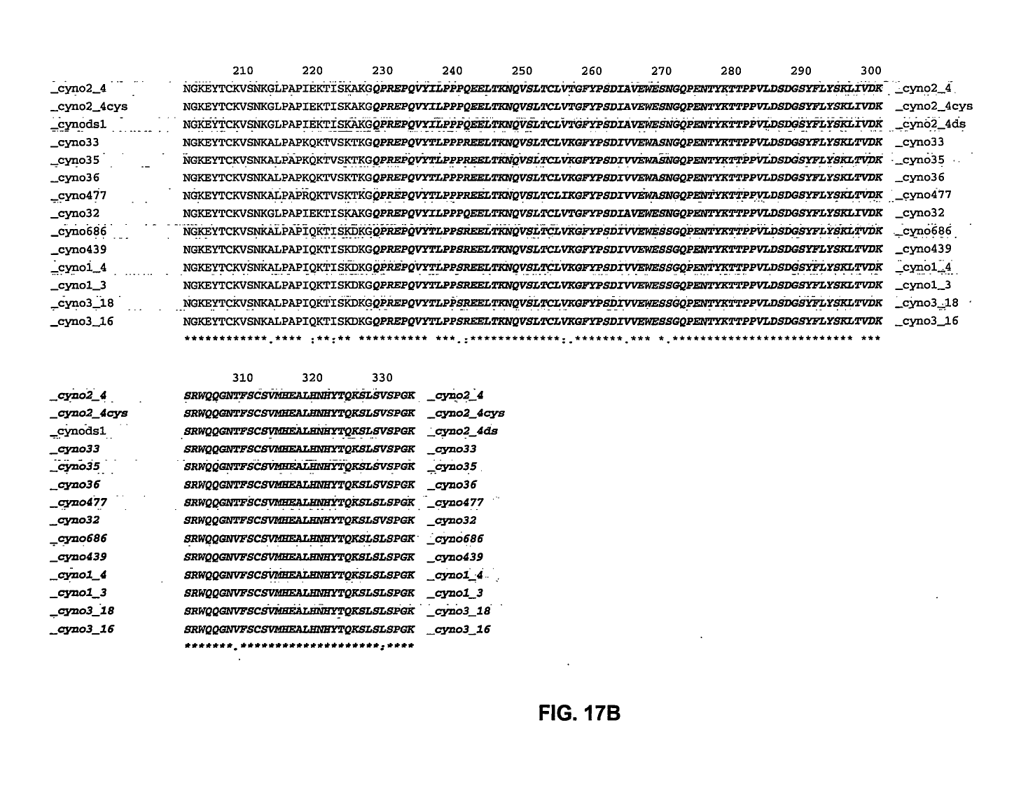

Additional heavy chain constant regions can be isolated according to the general procedures discussed above. Clones cyno3-16, cyno2-4, cyno33, cyno2-4cys, cynods1, and additional clones prepared by methods like those described above may be compared for similarities in nucleotide sequence and amino acid sequence. See, e.g., FIGS. 16 and 17.

### Example 2

### Cloning the Light Chain Constant Region from Cynomolgus Monkey Sequences

The native polynucleotides encoding cynomolgus monkey light chain kappa constant region is cloned from a cynomolgus monkey B cell cell line. RNA is isolated from the cell line and cDNA is synthesized from the RNA. The cDNA is used as a template for PCR with the following primers: 5′-ATCAAACGAGCTGTGGCTGCACCA-3′ (SEQ ID NO:45) and 5′-CAGGTGGGGGCACTTCTCCCT-3′ (SEQ ID NO:46). PCR is performed using an Expand High Fidelity PCR System (Roche) with the addition of 5% DMSO. Samples are first incubated for 2 minutes at 94° C., followed by 40 cycles under the following conditions per cycle: 30 seconds at 94° C.; 30 seconds at 45° C.; and 1 minute at 72° C. Samples are then incubated for 7 minutes at 72° C. following the last PCR cycle. PCR primers are used at a concentration of 30 pmol and 2 μl cDNA preparation is used.

Following the initial cloning, the polynucleotide encoding the cynomolgus monkey kappa constant region is constructed as a BssHII-NotI cassette by PCR. Nucleotide modifications are made at the 5′ end of the constant region to introduce a BssHII site. This does not alter the amino acid sequence. A NotI site is introduced 3′ to the termination codon.

### Example 3

### Assembling the Chimeric Heavy Chain and Light Chain and Production of Chimeric Antibodies

Chimeric Heavy Chain

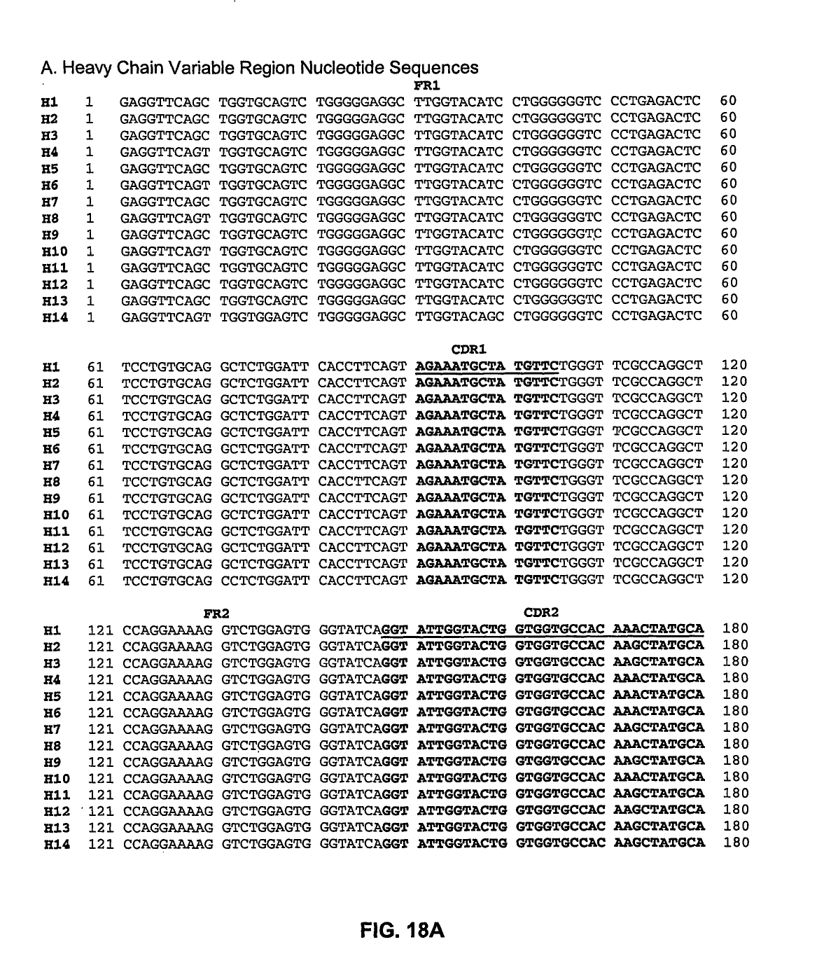

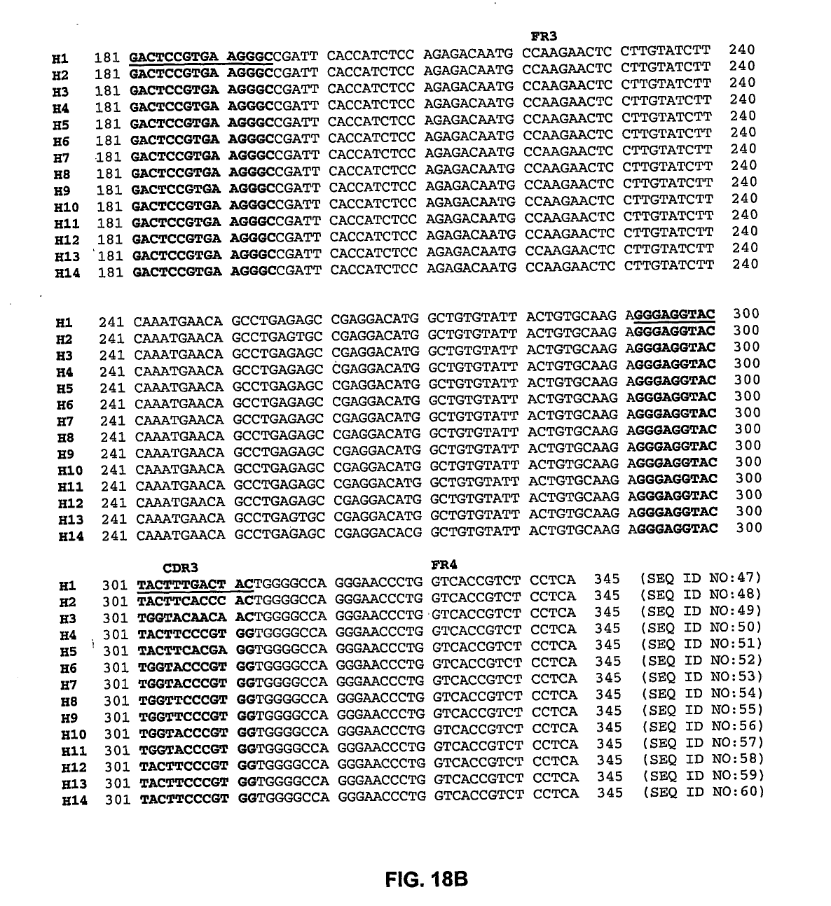

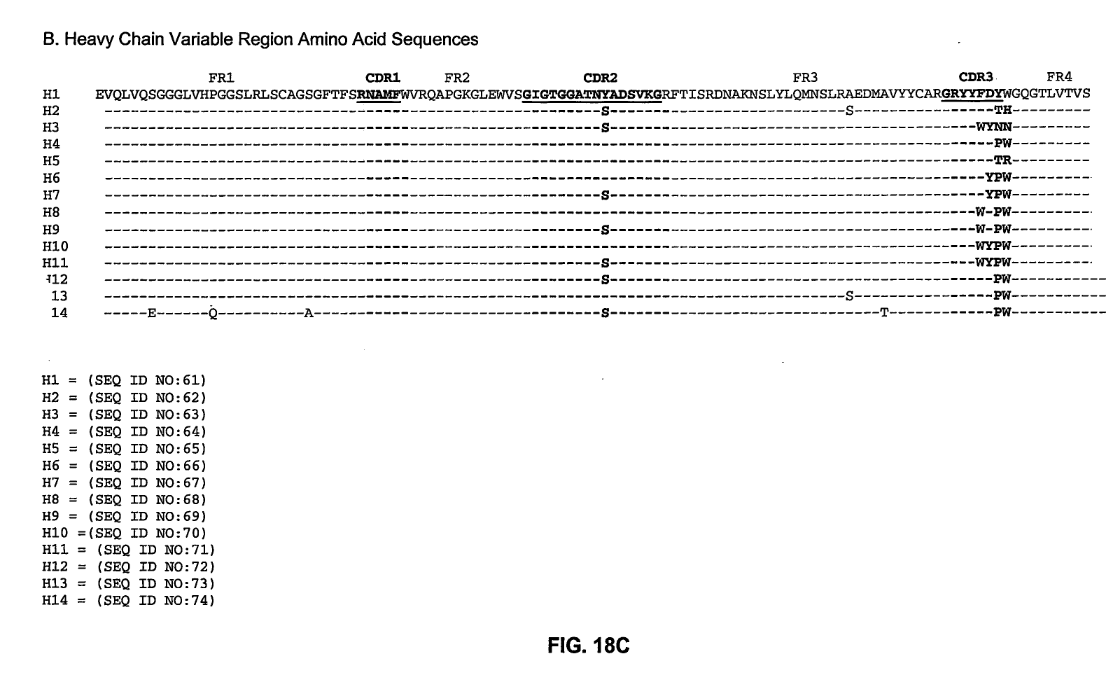

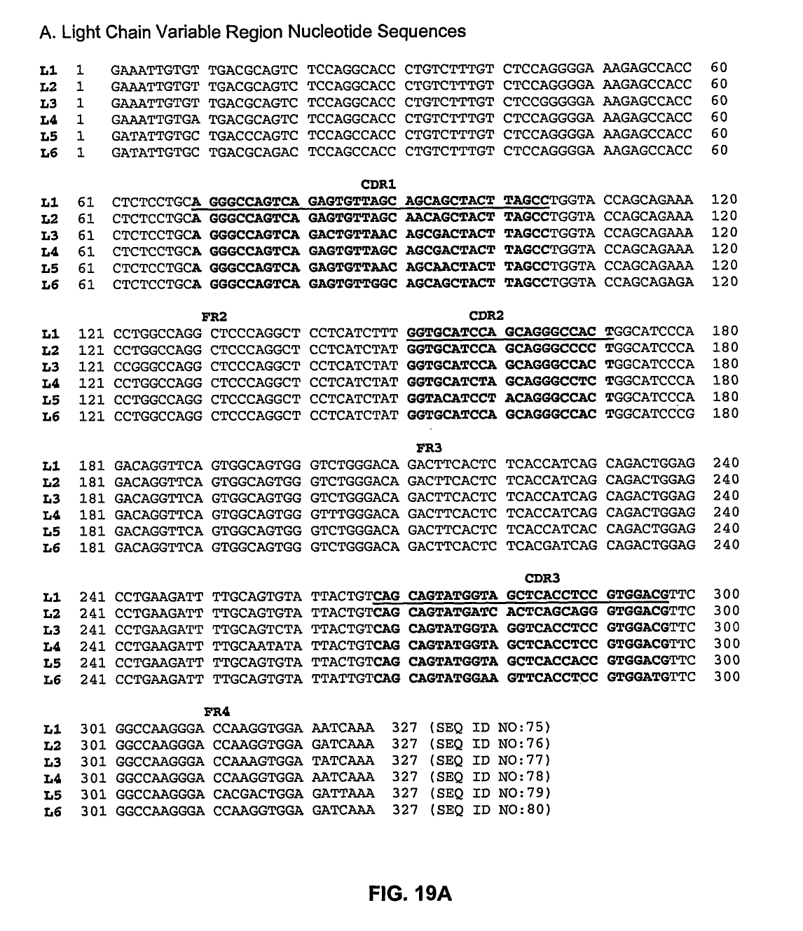

A full heavy chain molecule comprising a heavy chain variable region and a cynomolgus monkey constant region are made. Polynucleotide encoding the variable region is synthesized by PCR to generate either a SalI-NheI cassette or as a SalI-ApaI cassette. Both cassettes include Kozak and leader sequences 5′ of the sequence encoding the variable region. The 3′ end of the SalI-ApaI cassette include the nucleotides encoding the first 5 amino acids of the cynomolgus monkey constant region. Certain exemplary chimeric heavy chains can be produced with the heavy chain variable sequences provided in FIG. 18.

To make cyno3-16 heavy chain plasmid, the SalI-ApaI variable region cassette is attached to the cynomolgus monkey constant region cassettes described in Example 1A at the ApaI site located five amino acids from the beginning of the constant regions. The formed construct is cloned between the SalI and NotI sites of the transient expression vector pDC414-N.

To make cyno33 heavy chain plasmid, the SalI-ApaI variable region cassette is attached to the cynomolgus monkey constant region cassettes described in Example 1B at the ApaI site located five amino acids from the beginning of the constant regions. The formed construct is cloned between the SalI and NotI sites of the transient expression vector pDC414-N.

To make the cyno2-4 heavy chain plasmid, the SalI-NheI variable region cassette is attached to the polynucleotide encoding the cynomolgus monkey constant region cassettes as described in Example 1B at the NheI site. The formed construct is also cloned between the SalI and NotI sites of pDC414-N.

To make the cynodsl heavy chain plasmid, the SalI-NheI variable region cassette is attached to the polynucleotide encoding the cynomolgus monkey constant region cassettes as described in Example 1D at the NheI site. The formed construct is also cloned between the SalI and NotI sites of pDC414-N.

pDC414-N is a modified version of pDC412 (Ettehadieh et al., Cytotechnology 38:11-14 (2002)). PDC414-N contains a minimal 120 base pair Epstein-Barr replication origin (Shirakata and Hirai, J. Biochem. 123:175-181 (1998)) in place of the 2.1 kilobase pair Epstein-Barr replication origin in pDC412. The NheI site is also removed from the vector backbone of pDC414-N.

To make the cyno2-4cys heavy chain plasmid, the SalI-NheI variable region cassette is attached to the polynucleotide encoding the cyno2-4cys constant region describe in Example 1C at the NheI site. The formed construct is cloned between the SalI and NotI sites of the transient expression vector pDC409 (Girl et al., EMBO J. 13:2822-2830 (1994)).

Chimeric Light Chain

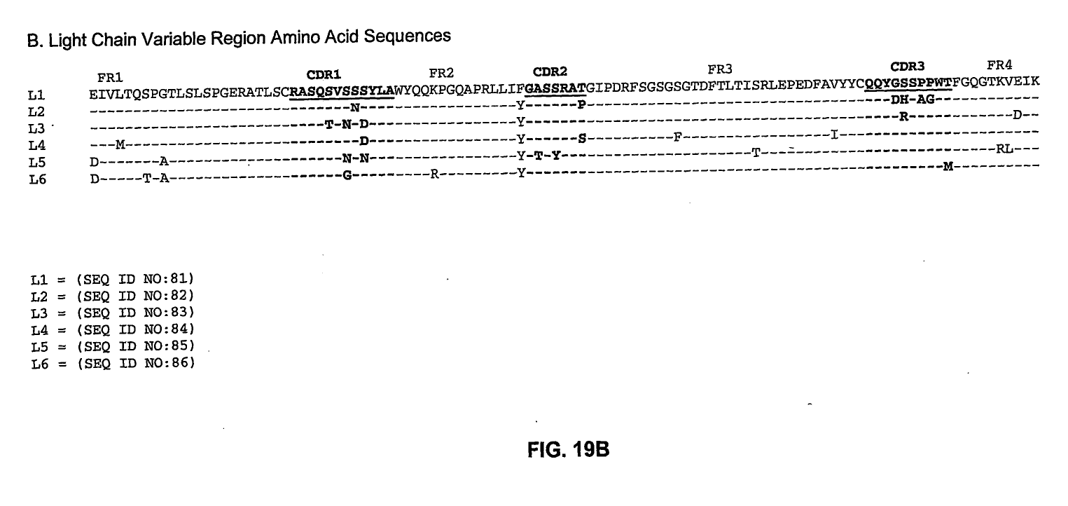

A full light chain molecule comprising a light chain variable region and a cynomolgus monkey constant region is made. The variable region is synthesized by PCR as a SalI-BssHII cassette. The cassette includes Kozak and leader sequences 5′ of the variable region. According to various embodiments, any light chain variable region from any species may be combined with an heavy chain constant region of a cynomolgus monkey. Certain exemplary chimeric light chains can be produced with the light chain variable sequences provided in FIG. 19.

To make the light chain plasmid, the SalI-BssHII variable region cassette is attached to the cynokappa NotI-BssHII constant region cassette described in Example 2 at the BssHII site. The resulting SalI-NotI cassette is cloned between the SalI and NotI sites of pDC414-N. Methods for constructing these chimeric heavy and lights chains entail enzyme digestion, ligation, and transformation into bacterial cell hosts according to procedures well known in the art.

Production of Chimelic Antibodies

The chimeric cynomolgus monkey heavy and light chain plasmids are co-transfected into E5 cells according to the methods of Ettehadieh et al. (Cytotechnology 38:11-14 (2002)) for transient expression of antibodies. Generally, cells are transfected using DEAE/dextran followed by a DMSO shock. Following transfection, the cells are grown for 7 days in low serum medium, containing 0.5% fetal bovine serum. Antibodies are purified from the cell supernatant.

The supernatant is passed over a 4.6×100 mm protein A resin column (POROS20 A from Perseptive Biosystems) at a flow rate of 10 ml/minute, after first equilibrating the column with PBS (Phosphate Buffered Saline pH 7.4). The flow-through is collected. The column is washed with 40 ml of PBS pH 7.4 and the protein is eluted using 15 ml of 0.1 M Glycine pH 2.7+0.3M NaCl collecting 15×1-ml fractions. The fractions are neutralized using 100 ul of 1.0 M Tris pH 8.0.

Samples are prepared using a protein 200 plus lab chip kit (Agilent) and are run on an Agilent 2100 bioanalyzer using the protein 200 assay, following manufacturer's instructions. For antibodies comprising cyno3-16, cyno33, and cyno2-4, 3 ul of PBS are mixed with 1 ul of antibody sample. For antibodies comprising cyno2-4cys and cynods1, 4 ul of antibody sample is used. This 4 ul is then mixed with 2 ul of denaturing, non-reducing solution. The samples are heated for 3 minutes at 100° C. and then diluted with 84 ul of distilled water. Six microliters of these diluted samples are applied to lab chips and analyzed for the presence of antibody.

Alternatively, samples can be analyzed on an SDS-PAGE gel, according to techniques standard in the art. The approximate molecular weight of a chimeric light chain is 23.3 kDa and the approximate molecular weight of a chimeric heavy chain is 49.7 kDa. On an SDS-PAGE gel, these molecular weights are approximately 29 kDa for the chimeric light chain and approximately 53 kDa for the chimeric heavy chain.

The fractions containing the antibody are transferred into PBS pH 7.2 or 6.8 using dialysis or an Amicon Centricon Plus 10 k MWCO filter unit (cat. no. UFC2LGC24) at 3000 RPM in a 4° C. centrifuge. After transfer to PBS the samples are sterile filtered with a 22 micron filter.

### Example 4

### Measuring Epitope Binding Ability of the Chimeric Antibody

To test the activity of the certain exemplary antibodies, they are used in activity assays to look for blocking of IL4 and IL13 induction of CD23 on B-cells. See, e.g., T. Defrance et al. (J Exp Med 165:1459 (1987)) and J. Punnonen et al. (Proc. Nat Acad. Sci. 90: 3730-34 (1993)) for a description of the induction of CD23 from B-cells by IL4 and IL13 respectively. In the activity assays, the antibodies are titrated into B-cell cultures containing IL4. Inhibition of CD23 expression is measured, for example, by FACS analysis using a fluorescent antibody to detect cell surface CD23.

### Example 5

### Measuring Fc Binding Ability of the Chimeric Antibody

Chimeric antibodies are titrated in PBS from 20 mg/ml (nM) in 2-fold dilutions, diluted 6 times, and pre-incubated with excess (1 mM) biotinylated soluble hulL-4R (made with aminohexanoyl-Biotin-N-hydroxy-succiniimide ester; Zymed cat. no. 004302) at 4° C. for 30 minutes. Biotinylated soluble huIL-4R is made with aminohexanoyl-Biotin-N-hydroxysucciniimide ester (Zymed cat. no. 004302) according to the manufacturer's instructions. This assay is adaptable for use with any chimeric antibody and the antigen it recognizes. T-depleted peripheral blood mononuclear cells (PBMC) are incubated in serum-free RPMI for 1 hour at 37° C. to allow shedding of FcR-bound cytophilic IgG. Cells are then stained with the titrated complexed anti-hulL-4R ab/biotin-huIL-4R. Cells are washed 2× in PBS, spinning at 150×g. The cells are then incubated with streptavidin-phycoerythrin (Molecular Probes, cat. no. S-866), which is diluted at 1:150 in PBS, at 4° C. for 30 minutes. Cells are washed 2× in PBS, at 150 ×g, and complex binding is detected by flow cytometric analysis gating on monocytes by size.

We claim:

1. An isolated polypeptide comprising an amino acid sequence as set forth in SEQ ID NO:6; SEQ ID NO:8; SEQ ID NO:10; SEQ ID NO:12; SEQ ID NO:14; or SEQ ID NO:20 and further comprising an antibody heavy chain variable region.

2. The isolated polypeptide of claim 1, wherein the antibody heavy chain variable region is a cynomolgus monkey antibody heavy chain variable region.

3. The isolated polypeptide of claim 1, wherein the antibody heavy chain variable region is an antibody heavy chain variable region of a species other than a cynomolgus monkey.

4. The isolated polypeptide of claim 1, wherein the antibody heavy chain variable region is a human antibody heavy chain variable region.

5. The isolated polypeptide of claim 1, wherein the antibody heavy chain variable region is a mouse antibody heavy chain variable region.

6. An isolated polypeptide comprising an amino acid sequence as set forth in SEQ ID NO:30 and further comprising an antibody light chain variable region.

7. The isolated polypeptide of claim 6, wherein the antibody light chain variable region is a cynomolgus monkey antibody light chain variable region.

8. The isolated polypeptide of claim 6, wherein the antibody light chain variable region is an antibody light chain variable region of a species other than a cynomolgus monkey.

9. The isolated polypeptide of claim 6, wherein the antibody light chain variable region is a human antibody light chain variable region.

10. The isolated polypeptide of claim 6, wherein the antibody light chain variable region is a mouse antibody light chain variable region.

11. An isolated polynucleotide comprising a sequence encoding a polypeptide comprising an amino acid sequence as set forth in SEQ ID NO:6; SEQ ID NO:8; SEQ ID NO:10; SEQ ID NO:12; SEQ ID NO:14; or SEQ ID NO:20 and further comprising a sequence encoding a polypeptide comprising an antibody heavy chain variable region.

12. The isolated polynucleotide of claim 11, wherein the sequence encoding a polypeptide comprising an antibody heavy chain variable region is a sequence encoding a cynomolgus monkey heavy chain variable region.

13. The isolated polynucleotide of claim 11, wherein the sequence encoding a polypeptide comprising an antibody heavy chain variable region is a sequence encoding an antibody heavy chain variable region of a species other than a cynomolgus monkey.

14. The isolated polynucleotide of claim 11, wherein sequence encoding a polypeptide comprising an antibody heavy chain variable region is a sequence encoding a human antibody heavy chain variable region.

15. The isolated polynucleotide of claim 11, wherein the sequence encoding a polypeptide comprising an antibody heavy chain variable region is a sequence encoding a mouse antibody heavy chain variable region.

16. An isolated polynucleotide comprising a sequence encoding a polypeptide comprising an amino acid sequence as set forth in SEQ ID NO:30 and further comprising a sequence encoding a polypeptide comprising an antibody light chain variable region.

17. The isolated polynucleotide of claim 16, wherein the sequence encoding a polypeptide comprising an antibody light chain variable region is a sequence encoding a cynomolgus monkey antibody light chain variable region.

18. The isolated polynucleotide of claim 16, wherein the sequence encoding a polypeptide comprising an antibody light chain variable region is a sequence encoding an antibody light chain variable region of a species other than a cynomolgus monkey.

19. The isolated polynucleotide of claim 16, wherein the sequence encoding a polypeptide comprising an antibody light chain variable region is a sequence encoding a human antibody light chain variable region.

20. The isolated polynucleotide of claim 16, wherein the sequence encoding a polypeptide comprising an antibody light chain variable region is a sequence encoding a mouse antibody light chain variable region.

21. The isolated polynucleotide of claim 11, wherein the isolated polynucleotide,comprises a nucleotide sequence as set forth in SEQ ID NO:5; SEQ ID NO:7; SEQ ID NO:9; SEQ ID NO:11; or SEQ ID NO:13; or SEQ ID NO:19.

22. The isolated polynucleotide of claim 16, wherein the isolated polynucleotide comprises a nucleotide sequence as set forth in SEQ ID NO:29.

23. An isolated antibody comprising a first polypeptide comprising an amino acid sequence as set forth in SEQ ID NO:6; SEQ ID NO:8; SEQ ID NO:10; SEQ ID NO:12; SEQ ID NO:14; or SEQ ID NO:20 and a second polypeptide comprising an amino acid sequence as set forth in SEQ ID NO:30.

24. The isolated antibody of claim 23, further comprising a cynomolgus monkey heavy chain variable region and a cynomolgus monkey light chain variable region.

25. The isolated antibody of claim 23, further comprising a heavy chain variable region of a species other than a cynomolgus monkey and a light chain variable region of the species other than a cynomolgus monkey.

26. The isolated antibody of claim 25, wherein the heavy chain variable region and the light chain variable region are from the same species.

27. The isolated antibody of claim 23, further comprising a human antibody heavy chain variable region and a human antibody light chain variable region.

28. The isolated antibody of claim 23, further comprising a mouse antibody heavy chain variable region and a mouse antibody light chain variable region.

29. A method for evaluating the effects of an antibody comprising:
a) introducing into a cynomolgus monkey a chimeric antibody comprising light chain and heavy chain variable regions from an antibody and light chain and heavy chain constant regions from a cynomolgus monkey; and
b) evaluating the effects of the chimeric antibody in the cynomolgus monkey.

30. The method of claim 29, wherein the evaluating comprises evaluating the efficacy of the chimeric antibody for treating or preventing a disease in the monkey.

31. The method of claim 29, wherein the evaluating comprises detecting an adverse event in the monkey.

32. An expression vector comprising an isolated polynucleotide of claim 11.

33. An expression vector comprising an isolated polynucleotide of claim 16

34. A cell comprising at least one of the expression vectors of claim 32 or claim 33.

35. A method of making a polypeptide comprising:
a) incubating a cell comprising the expression vector of claim 32 in conditions suitable for expression of the polypeptide; and
b) isolating the polypeptide.

36. A method of making a polypeptide comprising:
a) incubating a cell comprising the expression vector of claim 33 in conditions suitable for expression of the polypeptide; and
b) isolating the polypeptide.

37. A method of making a chimeric antibody comprising:
a) incubating a cell comprising the expression vector of claim 32 and further comprising the expression vector of claim 33 in conditions suitable for expression of the chimeric antibody; and
b) isolating the chimeric antibody.
